(19) United States
(12) Patent Application Publication
(10) Pub. No.: US 2025/0133617 A1
(43) Pub. Date: Apr. 24, 2025

(54) SYSTEM AND METHODS FOR WIRELESS CONNECTION IN AN ANALYTE MONITORING SYSTEM

(71) Applicant: Dexcom, Inc., San Diego, CA (US)

(72) Inventor: Javaid MASOUD, San Diego, CA (US)

(21) Appl. No.: 18/919,368

(22) Filed: Oct. 17, 2024

Related U.S. Application Data
(60) Provisional application No. 63/591,925, filed on Oct. 20, 2023.

(51) Int. Cl. H04W 76/14; A61B 5/00; H04W 24/08; H04W 84/18

(52) U.S. Cl. CPC H04W 76/14; A61B 5/0004; A61B 5/002; H04W 24/08; H04W 84/18

(57) ABSTRACT

Aspects of the present disclosure provide techniques for avoiding or reducing signal loss events between an analyte sensor system and a display device in an analyte monitoring system. An example method performed by the display device includes establishing a short-range wireless connection with an analyte sensor system, receiving one or more information packets from the analyte sensor system, determining a signal strength associated with the short-range wireless connection based on the one or more information packets received from the analyte sensor system, and providing, to a user of the analyte sensor system, an indication of the signal strength associated with the short-range wireless connection.

### CROSS-REFERENCE TO RELATED APPLICATIONS

This application claims priority to and benefit of U.S. Provisional Patent Application No. 63/591,925, filed Oct. 20, 2023, which is hereby expressly incorporated by reference herein in its entirety as if fully set forth below and for all applicable purposes.

### TECHNICAL FIELD

The present disclosure relates generally to an electronic device, such as an analyte sensor system for monitoring analyte values of a user.

### BACKGROUND

Diabetes is a metabolic condition relating to the production or use of insulin by the body. Insulin is a hormone that allows the body to use glucose for energy, or store glucose as fat. When a person cats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher-than-normal blood sugar level is called “hyperglycemia.” Chronic hyperglycemia can lead to several of health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce adequate insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

### SUMMARY

Aspects of the present disclosure provide a method for communication by a display device in an analyte monitoring system. The method includes establishing a short-range wireless connection with an analyte sensor system, receiving one or more information packets from the analyte sensor system, determining a signal strength associated with the short-range wireless connection based on the one or more information packets received from the analyte sensor system, and providing, to a user of the analyte sensor system, an indication of the signal strength associated with the short-range wireless connection.

Additional aspects of the present disclosure provide an analyte monitoring system. The analyte monitoring system comprises an analyte sensor system and a display device. The analyte sensor system is configured to: measure analyte levels associated with a user of the analyte sensor system and transmit one or more information packets to the display device. The display device is configured to: establish a short-range wireless connection with the analyte sensor system, receive the one or more information packets from the analyte sensor system, determine a signal strength associated with the short-range wireless connection based on the one or more information packets received from the analyte sensor system, and provide, to a user of the analyte sensor system, an indication of the signal strength associated with the short-range wireless connection.

Additional aspects of the present disclosure provide a processing system for communication by a display device in an analyte monitoring system. The processing system includes one or more processors individually or collectively configured to execute instructions stored on one or more memories and to cause the display device to: establish a short-range wireless connection with an analyte sensor system, receive one or more information packets from the analyte sensor system, determine a signal strength associated with the short-range wireless connection based on the one or more information packets received from the analyte sensor system, and provide, to a user of the analyte sensor system, an indication of the signal strength associated with the short-range wireless connection.

### BRIEF DESCRIPTION OF THE DRAWINGS

Further aspects of the present disclosure will be more readily appreciated upon review of the detailed description of the various disclosed embodiments, described below, when taken in conjunction with the accompanying figures.

FIG. 1 illustrates aspects of an example system that may be used in connection with some embodiments.

FIG. 2 illustrates aspects of an example system that may be used in connection with some embodiments.

FIG. 3A is an example analyte sensor system, in accordance with some embodiments.

FIG. 3B is an example analyte sensor system, in accordance with some embodiments.

FIG. 4 illustrates aspects of an example analyte sensor system, in accordance with some embodiments.

FIG. 5 illustrates aspects of an example analyte sensor system, in accordance with some embodiments.

FIG. 6 depicts a process flow including operations for communications in an analyte monitoring system between a display device and an analyte sensor system, in accordance with some embodiments.

FIG. 7 illustrates an example of providing an indication of a signal strength of a Bluetooth connection as a signal strength indicator represented as a number of bars, in accordance with some embodiments.

FIG. 8 illustrates an example of providing an indication of the signal strength of the Bluetooth connection as a notification of a signal strength event, in accordance with some embodiments.

FIG. 9 illustrates a method by a display device, in accordance with some embodiments.

FIG. 10 illustrates a communications device, in accordance with some embodiments.

The figures, described in greater detail in the description and examples below, are provided for purposes of illustration only, and merely depict typical or example embodiments of the disclosure. The figures are not intended to be exhaustive or to limit the disclosure to the precise form disclosed. It should also be understood that the disclosure may be practiced with modification or alteration, and that the disclosure may be limited only by the claims and the equivalents thereof.

### DETAILED DESCRIPTION

Aspects of the present disclosure provide systems, methods, and devices for reducing or avoiding signal loss events associated with a short-range wireless connection, such as Bluetooth (e.g., Bluetooth classic, Bluetooth Low Energy (BLE), etc.) or other short-range wireless connections, between an analyte sensor system and a display device. For example, analyte sensor systems may be worn by users and are configured to continuously measure analyte levels of the user. These analyte levels may then be transmitted from the analyte sensor system to a display device (e.g., smart phone) using an antenna system comprising one or more antennas, allowing the user to conveniently track their analyte levels.

In some embodiments, information packets including the analyte levels of the user may be transmitted from the analyte sensor system to the display device using a short-range wireless connection established between the analyte sensor system and the display device. Proper reception of the information packets by the display device requires that these information packets be received with a strong signal strength. If a signal strength associated with transmissions on the short-range wireless connection is weak or intermittent, this may lead to the information packets being lost or corrupted.

The signal strength associated with the short-range wireless connection established between the analyte sensor system and display device may be negatively affected by various factors, such as interference from other devices, a user of the analyte sensor system setting the display device down and walking a far distance away, the user placing the analyte sensor system or display device in a particular position or location that blocks the transmission/reception of the information packets, and the like. Each scenario may lead to a reduction in signal strength associated with the short-range wireless connection and may result in lost packets, which may in turn lead to a decrease in battery life of the analyte sensor system (e.g., associated with retransmissions of the lost packets) or the user not being provided their analyte levels.

Accordingly, aspects of the present disclosure provide techniques for helping to avoid or reduce signal loss events associated with the short-range wireless connection between an analyte sensor system and display device. In some embodiments, these techniques may include providing, to the user of the analyte sensor system, an indication of a signal strength of a short-range wireless connection between the analyte sensor system and display device, allowing the user to take preventative action to avoid or reduce these signal loss events. The techniques for helping to avoid or reduce signal loss events may also include corrective actions, such as automatically increasing a transmission power. Increasing the transmission power may increase a transmission range between the analyte sensor system and display device, avoiding or reducing signal loss events and lost packets.

By avoiding or reducing these signal loss events, a connection between the analyte sensor system and display device may be continuously maintained, allowing the user to be continuously informed regarding their analyte levels. Additionally, avoiding or reducing these signal loss events may reduce a number of lost or corrupted packets, thereby reducing a number of retransmissions of packets between the analyte sensor system and display device, and reducing power consumption.

The details of some example embodiments of the systems, methods, and devices of the present disclosure are set forth in this description and in some cases, in other portions of the disclosure. Other features, objects, and advantages of the disclosure will be apparent to one of skill in the art upon examination of the present disclosure, description, figures, examples, and claims. It is intended that all such additional systems, methods, devices, features, and advantages be included within this description (whether explicitly or by reference), be within the scope of the present disclosure, and be protected by one or more of the accompanying claims.

### System Overview and Example Configurations

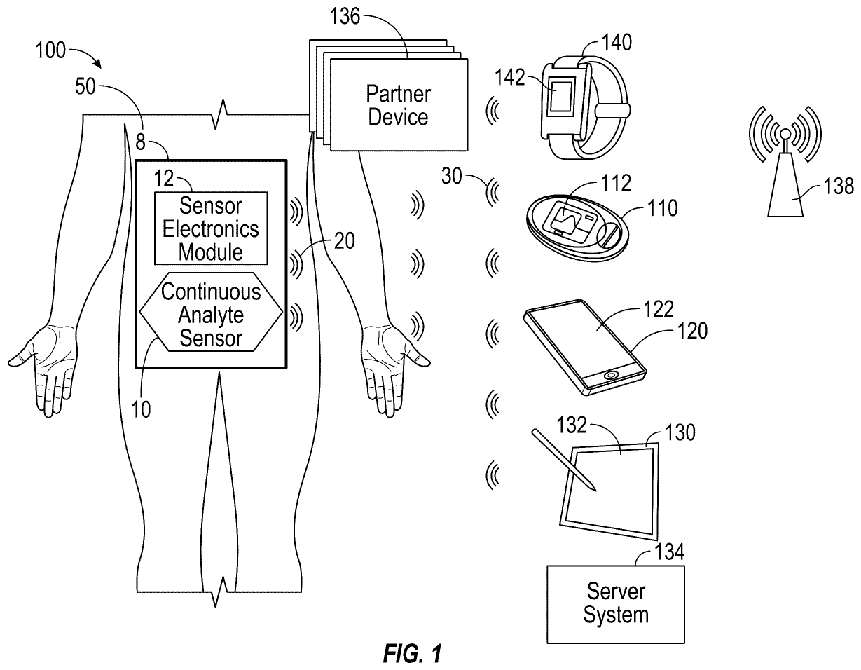

FIG. 1 depicts an analyte monitoring system 100 that may be used in connection with embodiments of the present disclosure that involve gathering, monitoring, and/or providing information regarding analyte values present in a user's body, including for example the user's blood glucose values, other analytes, multiple multiplexed or simultaneous measured analytes, or the like. Analyte monitoring system 100 depicts aspects of analyte sensor system 8 that may be communicatively coupled to display devices 110, 120, 130, and 140, partner devices 136, and/or server system 134.

Analyte sensor system 8 in the illustrated embodiment includes analyte sensor electronics module 12 and analyte sensor 10 associated with analyte sensor electronics module 12. Analyte sensor electronics module 12 may be electrically and mechanically coupled to analyte sensor 10 before analyte sensor 10 is implanted in a user or host. Accordingly, analyte sensor 10 may not require a user to couple analyte sensor electronics module 12 to analyte sensor 10. For example, analyte sensor electronics module 12 may be physically/mechanically and electrically coupled to analyte sensor 10 during manufacturing, and this physical/mechanical and electrical connection may be maintained during shipping, storage, insertion, use, and removal of analyte sensor system 8. As such, the electro-mechanically connected components (e.g., analyte sensor 10 and analyte sensor electronics module 12) of analyte sensor system 8 may be referred to as a “pre-connected” system.

Analyte sensor electronics module 12 may be in wireless communication (e.g., directly or indirectly) with one or more of display devices 110, 120, 130, and 140. In addition, or alternatively to display devices 110, 120, 130, and 140, analyte sensor electronics module 12 may be in wireless communication (e.g., directly or indirectly) with partner devices 136 and/or server system 134. Likewise, in some examples, display devices 110-140 may additionally or alternatively be in wireless communication (e.g., directly or indirectly) with partner devices 136 and/or server system 134. Various couplings shown in FIG. 1 can be facilitated with wireless access point (WAP) 138, as also mentioned below.

In certain embodiments, analyte sensor electronics module 12 includes electronic circuitry associated with measuring and processing analyte sensor data or information, including prospective algorithms associated with processing and/or calibration of the analyte sensor data/information. Analyte sensor electronics module 12 can be physically/mechanically connected to analyte sensor 10 and can be integral with (non-releasably attached to) or releasably attachable to analyte sensor 10. Analyte sensor electronics module 12 may also be electrically coupled to analyte sensor 10, such that the components may be electromechanically coupled to one another.

Analyte sensor electronics module 12 may include hardware, firmware, and/or software that enables measurement and/or estimation of levels of the analyte in a host/user via analyte sensor 10 (e.g., which may be/include a glucose sensor). For example, analyte sensor electronics module 12 can include one or more of a potentiostat, a power source for providing power to analyte sensor 10, other components useful for signal processing and data storage, and a telemetry module for transmitting data from the sensor electronics module to one or more display devices. Electronics can be affixed to a printed circuit board (PCB) within analyte sensor system 8, or platform or the like, and can take a variety of forms. For example, the electronics can take the form of an integrated circuit (IC), such as an Application-Specific Integrated Circuit (ASIC), a microcontroller, a processor, and/or a state machine.

Analyte sensor electronics module 12 may include sensor electronics that are configured to process sensor information, such as sensor data, and generate transformed sensor data and displayable sensor information. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entireties.

With further reference to FIG. 1, display devices 110, 120, 130, and/or 140 can be configured for displaying (and/or alarming) displayable sensor information that may be transmitted by analyte sensor electronics module 12 (e.g., in a customized data package that is transmitted to the display devices based on their respective preferences). Each of display devices 110, 120, 130, or 140 can (respectively) include a display such as touchscreen display 112, 122, 132,/or 142 for displaying sensor information and/or analyte data to a user and/or receiving inputs from the user. For example, a graphical user interface (GUI) may be presented to the user for such purposes. In embodiments, the display devices may include other types of user interfaces such as voice user interface instead of or in addition to a touchscreen display for communicating sensor information to the user of the display device and/or receiving user inputs. In embodiments, one, some, or all of display devices 110, 120, 130, 140 may be configured to display or otherwise communicate the sensor information as it is communicated from analyte sensor electronics module 12 (e.g., in a data package that is transmitted to respective display devices), without any additional prospective processing required for calibration and/or real-time display of the sensor data.

The plurality of display devices 110, 120, 130, 140 depicted in FIG. 1 may include a custom display device, for example, analyte display device 110, specially designed for displaying certain types of displayable sensor information associated with analyte data received from analyte sensor electronics module 12 (e.g., a numerical value and/or an arrow, in embodiments). In embodiments, one of the plurality of display devices 110, 120, 130, 140 includes a smartphone, such as a mobile phone, based on an Android, iOS, or other operating system, and configured to display a graphical representation of the continuous sensor data (e.g., including current and/or historic data).

As further illustrated in FIG. 1 and mentioned above, analyte monitoring system 100 may also include WAP 138 that may be used to couple one or more of analyte sensor system 8, the plurality display devices 110, 120, 130, 140 etc., server system 134, and partner devices 136 to one another. For example, WAP 138 may provide WiFi and/or cellular or other wireless connectivity within analyte monitoring system 100. Near Field Communication (NFC) may also be used among devices of analyte monitoring system 100 for exchanging data, as well as for performing specialized functions, e.g., waking up or powering a device or causing the device (e.g., analyte sensor electronics module 12 and/or a transmitter) to exit a lower power mode or otherwise change states and/or enter an operational mode. Server system 134 may be used to collect analyte data from analyte sensor system 8 and/or the plurality of display devices, for example, to perform analytics thereon, generate universal or individualized models for glucose levels and profiles, provide services or feedback, including from individuals or systems remotely monitoring the analyte data, and so on.

Partner device(s) 136, by way of overview and example, can usually communicate (e.g., wirelessly) with analyte sensor system 8, including for authentication of partner device(s) 136 and/or analyte sensor system 8, as well as for the exchange of analyte data, medicament data, other data, and/or control signaling or the like. Partner devices 136 may include a passive device in example embodiments of the disclosure. One example of partner device 136 may be an insulin pump for administering insulin to a user in response and/or according to an analyte level of the user as measured/approximated using analyte sensor system 8. For a variety of reasons, it may be desirable for such an insulin pump to receive and track glucose values transmitted from analyte sensor system 8 (with reference to FIG. 1 for example). One example reason for this is to provide the insulin pump a capability to suspend/activate/control insulin administration to the user based on the user's glucose value being below/above a threshold value.

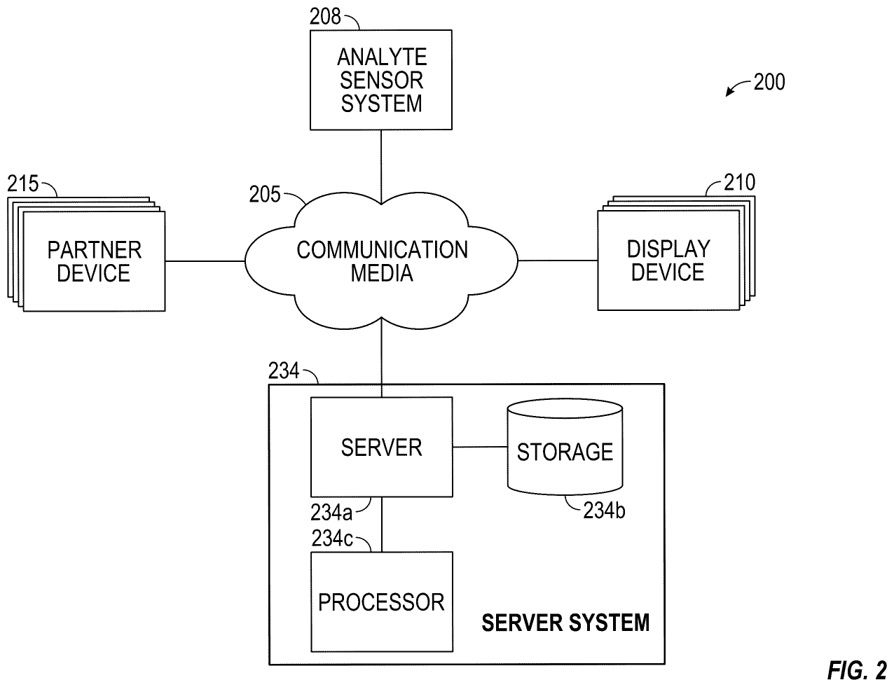

Referring now to FIG. 2, health monitoring and management system 200 is depicted. System 200 may be used in connection with implementing embodiments of the disclosed systems, methods, apparatuses, and/or devices, including, for example, aspects described above in connection with FIG. 1. By way of example, various below-described components of FIG. 2 may be used to enable wireless communication of analyte (e.g., glucose) data, for example among/between analyte sensor system 208, display devices 210, partner devices 215, and/or one or more server systems 234, and so on. In some cases, analyte sensor system 208 illustrated in FIG. 2 may be an example of the analyte sensor system 8 illustrated in FIG. 1. Additionally, in some cases, the display devices 210 illustrated in FIG. 2 may be examples of the display devices 110, 120, 130, and 140 illustrated in FIG. 1. Additionally, in some cases, partner devices 215 illustrated in FIG. 2 may be examples of the partner device 136 illustrated in FIG. 1.

As shown in FIG. 2, system 200 may include analyte sensor system 208, one or more display devices 210, and/or one or more partner devices 215. Additionally, in the illustrated embodiment, system 200 includes server system 234, which can in turn include server 234a coupled to processor 234c and storage 234b. Analyte sensor system 208 may be coupled to display devices 210, partner devices 215, and/or server system 234 via communication media 205. Some details of the processing, gathering, and exchanging of data, and/or executing actions (e.g., providing medicaments or related instructions) by analyte sensor system 208, partner devices 215, and/or display device 210, etc., are provided below. Herein, display devices 210, partner devices 215, and server system 234 may be referred to as communications devices and may be configured to communicate with analyte sensor system 208.

Analyte sensor system 208, display devices 210, and/or partner devices 215 may exchange messaging (e.g., control signaling) via communication media 205, and communication media 205 may also be used to deliver analyte data to display devices 210, partner devices 215, and/or server system 234. As alluded to above, display devices 210 may include a variety of electronic computing devices, such as a smartphone, tablet, laptop, wearable device, etc. Display devices 210 may also include analyte display device 110 that may be customized for the display and conveyance of analyte data and related notifications etc. Partner devices 215 may include medical devices, such as an insulin pump or pen, connectable devices, such as a smart fridge or mirror, key fob, and other devices.

In certain embodiments, communication media 205 may implemented using one or more wireless communication protocols, such as for example Bluetooth, Bluetooth Low Energy (BLE), ZigBee, WiFi, IEEE 802.11 protocols, Infrared (IR), Radio Frequency (RF), 2G, 3G, 4G, 5G, etc., and/or wired protocols and media. It will also be appreciated upon studying the present disclosure that communication media can be implemented as one or more communication links, including in some cases, separate links, between the components of system 200, whether or not such links are explicitly shown in FIG. 2 or referred to in connection therewith. By way of illustration, analyte sensor system 208 may be coupled to display device 210 via a first link of communication media 205 using BLE, while analyte sensor system 208 may be coupled to server system 234 by a second link of communication media 205 using a WiFi communication protocol. In embodiments, a BLE signal may be temporarily attenuated to minimize data interceptions. For example, attenuation of a BLE signal through hardware or firmware design may occur temporarily during moments of data exchange (e.g., pairing).

In embodiments, the elements of system 200 may be used to perform operations of various processes described herein and/or may be used to execute various operations and/or features described herein with regard to one or more disclosed systems and/or methods. Upon studying the present disclosure, one of skill in the art will appreciate that system 200 may include single or multiple analyte sensor systems 208, communication media 205, and/or server systems 234.

As mentioned, communication media 205 may be used to connect or communicatively couple analyte sensor system 208, display devices 210, partner devices 215, and/or server system 234 to one another or to a network. Communication media 205 may be implemented in a variety of forms. For example, communication media 205 may include one or more of an Internet connection, such as a local area network (LAN), a person area network (PAN), a wide area network (WAN), a fiber optic network, internet over power lines, a hard-wired connection (e.g., a bus), DSL, and the like, or any other kind of network connection or communicative coupling. Communication media 205 may be implemented using any combination of routers, cables, modems, switches, fiber optics, wires, radio (e.g., microwave/RF, AM, FM links etc.), and the like. Upon reading the present disclosure, one of skill in the art will recognize other ways to implement communication media 205 for communications purposes and will also recognize that communication media 205 may be used to implement features of the present disclosure using as of yet undeveloped communications protocols that may be deployed in the future.

Further referencing FIG. 2, server 234a may receive, collect, and/or monitor information, including analyte data, medicament data, and related information, from analyte sensor system 208, partner devices 215 and/or display devices 210, such as input responsive to the analyte data or medicament data, or input received in connection with an analyte monitoring application running on analyte sensor system 208 or display device 210, or a medicament delivery application running on display device 210 or partner device 215. As such, server 234a may receive, collect, and/or monitor information from partner devices 215, such as, for example, information related to the provision of medicaments to a user and/or information regarding the operation of one or more partner devices 215. Server 234a may also receive, collect, and/or monitor information regarding a user of analyte sensor system 208, display devices 210, and/or partner devices 215.

In embodiments, server 234a may be adapted to receive such information via communication media 205. This information may be stored in storage 234b and may be processed by processor 234c. For example, processor 234c may include an analytics engine capable of performing analytics on information that server 234a has collected, received, etc. via communication media 205. In embodiments, server 234a, storage 234b, and/or processor 234c may be implemented as a distributed computing network, such as a Hadoop RTM network, or as a relational database or the like. The aforementioned information may then be processed at server 234a such that services may be provided to analyte sensor system 208, display devices 210, partner devices 215, and/or a user(s) thereof. For example, such services may include diabetes management feedback for the user.

Server 234a may include, for example, an Internet server, a router, a desktop or laptop computer, a smartphone, a tablet, a processor, a module, or the like, and may be implemented in various forms, including, for example, an integrated circuit or collection thereof, a printed circuit board or collection thereof, or in a discrete housing/package/rack or multiple of the same. In embodiments, server 234a at least partially directs communications made over communication media 205. Such communications may include the delivery of analyte data, medicament data, and/or messaging related thereto (e.g., advertisement, authentication, command, or other messaging). For example, server 234a may process and exchange messages between and/or among analyte sensor system 208, display devices 210, and/or partner devices 215 related to frequency bands, timing of transmissions, security/encryption, alarms, alerts, notifications, and so on.

Server 234a may update information stored on analyte sensor system 208, partner devices 215, and/or display devices 210, for example, by delivering applications thereto or updating the same, and/or by reconfiguring system parameters or other settings of analyte sensor system 208, partner devices 215, and/or display devices 210. Server 234a may send/receive information to/from analyte sensor system 208, partner devices 215, and/or display devices 210 in real time, periodically, sporadically, or on an event-drive basis. Further, server 234a may implement cloud computing capabilities for analyte sensor system 208, partner devices 215, and/or display devices 210.

With the above description of aspects of the presently disclosed systems and methods for wireless communication of analyte data, examples of some specific features of the present disclosure will now be provided. It will be appreciated by one of skill in the art upon studying the present disclosure that these features may be implemented using aspects and/or combinations of aspects of the example configurations described above, whether or not explicit reference is made to the same.

### Analyte Data

Referring back to FIG. 1, as mentioned above, in embodiments, analyte sensor system 8 is provided for measurement of an analyte in a host or user. By way of an overview and an example, analyte sensor system 8 may be implemented as an encapsulated microcontroller that makes sensor measurements, generates analyte data (e.g., by calculating values for continuous glucose monitoring data), and engages in wireless communications (e.g., via Bluetooth and/or other wireless protocols) to send such data to remote devices (e.g., display devices 110, 120, 130, 140, partner devices 136, and/or server system 134).

Analyte sensor system 8 may include analyte sensor 10 configured to measure a concentration or level of the analyte in the host, and analyte sensor electronics module 12 that is typically physically connected to analyte sensor 10 before analyte sensor 10 is implanted in a user. In some cases, the analyte sensor 10 may be a single-analyte sensor or a multi-analyte sensor capable of measuring one or more analytes, such as glucose, lactate, potassium, and the like. In embodiments, analyte sensor electronics module 12 includes electronics configured to process a data stream associated with an analyte concentration measured by analyte sensor 10, in order to generate sensor information, such as analyte data, that includes raw sensor data, transformed sensor data, and/or any other sensor data, for example. Analyte sensor electronics module 12 may further be configured to generate analyte sensor information that is customized for respective display devices 110, 120, 130, 140, partner devices 136, and/or server system 134. Analyte sensor electronics module 12 may further be configured such that different devices may receive different sensor information and may further be configured to wirelessly transmit sensor information to such display devices 110, 120, 130, 140, partner devices 136, and/or server system 134.

The term “analyte” as used herein is a broad term and is to be given its ordinary and customary meaning to a person of ordinary skill in the art (and is not to be limited to a special or customized meaning), and furthermore refers without limitation to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the sensor heads, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1,); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniac, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxinc-binding globulin; elements; trace transferring; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

### Analyte Sensor System

As described to above with reference to FIG. 1, in some embodiments, analyte sensor 10 includes a continuous glucose sensor, for example, a subcutaneous, transdermal (e.g., transcutaneous), or intravascular device. In embodiments, such a sensor or device can continuously measure and analyze glucose measurements in the interstitial fluid, blood samples, etc., depending on whether the device is subcutaneous, transdermal, or intravascular. Analyte sensor 10 can use any method of analyte measurement, including for example glucose-measurement, including enzymatic, chemical, physical, electrochemical, spectrophotometric, polarimetric, calorimetric, iontophoretic, radiometric, immunochemical, and the like.

In embodiments where analyte sensor 10 is a glucose sensor, analyte sensor 10 can use any method, including invasive, minimally invasive, and non-invasive sensing techniques (e.g., fluorescence monitoring), or the like, to provide a data stream indicative of the concentration or level of glucose in a host. The data stream may be a raw data signal, which may be converted into a calibrated and/or filtered analyte data that can be used to provide a useful value of glucose to a user, such as a patient or a caretaker (e.g., a parent, a relative, a guardian, a teacher, a doctor, a nurse, or any other individual that has an interest in the wellbeing of the host).

A glucose sensor may be any device capable of measuring the concentration of glucose. According to one example embodiment described below, an implantable glucose sensor may be used. However, it should be understood that the devices and methods described herein may be applied to any device capable of detecting a concentration of an analyte, glucose for example, and providing an output signal that represents the concentration of the analyte, again glucose for example (e.g., as a form of analyte data).

In embodiments, analyte sensor 10 is an implantable glucose sensor, such as described with reference to U.S. Pat. No. 6,001,067 and U.S. Patent Publication No. US-2005-0027463-A1. In embodiments, analyte sensor 10 is a transcutaneous glucose sensor, such as described with reference to U.S. Patent Publication No. US-2006-0020187-A1. In embodiments, analyte sensor 10 is configured to be implanted in a host vessel or extracorporeally, such as is described in U.S. Patent Publication No. US-2007-0027385-A1, co-pending U.S. Patent Publication No. US-2008-0119703-A1 filed Oct. 4, 2006, U.S. Patent Publication No. US-2008-0108942-A1 filed on Mar. 26, 2007, and U.S. Patent Application No. US-2007-0197890-A1 filed on Feb. 14, 2007. In embodiments, the continuous glucose sensor includes a transcutaneous sensor such as described in U.S. Pat. No. 6,565,509 to Say et al., for example.

In embodiments, analyte sensor 10 is a continuous glucose sensor that includes a subcutaneous sensor such as described with reference to U.S. Pat. No. 6,579,690 to Bonnecaze et al. or U.S. Pat. No. 6,484,046 to Say et al., for example. In embodiments, the continuous glucose sensor includes a refillable subcutaneous sensor such as described with reference to U.S. Pat. No. 6,512,939 to Colvin et al., for example. The continuous glucose sensor may include an intravascular sensor such as described with reference to U.S. Pat. No. 6,477,395 to Schulman et al., for example. The continuous glucose sensor may include an intravascular sensor such as described with reference to U.S. Pat. No. 6,424,847 to Mastrototaro et al., for example.

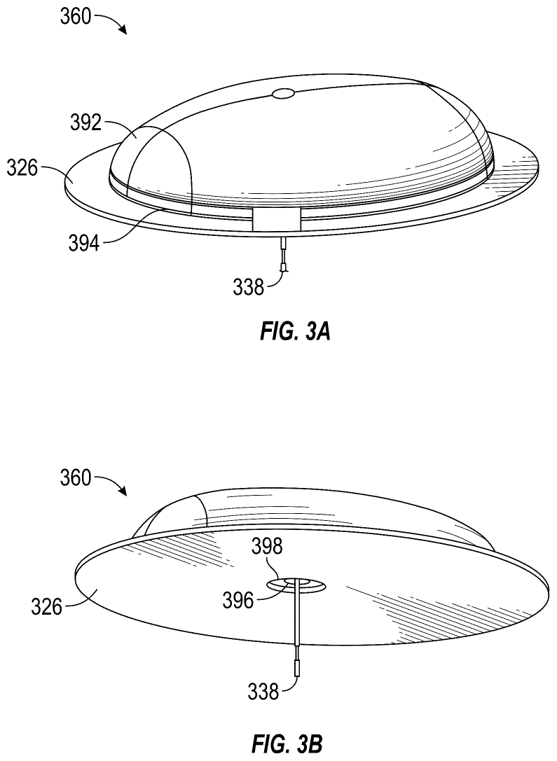

FIG. 3A illustrates a perspective view of an on-skin sensor assembly 360 that may be used in connection with the analyte sensor system 8 of FIG. 1 and/or the analyte sensor system 208 of FIG. 2. For example, on-skin sensor assembly 360 may be or include analyte sensor system 8 and/or analyte sensor system 208. On-skin sensor assembly 360 may include an outer housing with a first, top portion 392 and a second, bottom portion 394. In embodiments, the outer housing may include a clamshell design. On-skin sensor assembly 360 may include, for example, similar components as analyte sensor electronics module 12 described above in connection with FIG. 1, for example, a potentiostat, a power source for providing power to analyte sensor 10, signal processing components, data storage components, and a communication module (e.g., a telemetry module) for one-way or two-way data communication, a printed circuit board (PCB), an integrated circuit (IC), an Application-Specific Integrated Circuit (ASIC), a microcontroller, and/or a processor.

As shown in FIG. 3A, the outer housing may feature a generally oblong shape. The outer housing may further include aperture 396 disposed substantially through a center portion of outer housing and adapted for sensor 338 and needle insertion through a bottom of on-skin sensor assembly 360. In embodiments, aperture 396 may be a channel or elongated slot. On-skin sensor assembly 360 may further include an adhesive patch 326 configured to secure on-skin sensor assembly 360 to skin of the host. In embodiments, adhesive patch 326 may include an adhesive suitable for skin adhesion, for example a pressure sensitive adhesive (e.g., acrylic, rubber-based, or other suitable type) bonded to a carrier substrate (e.g., spun lace polyester, polyurethane film, or other suitable type) for skin attachment, though any suitable type of adhesive is also contemplated. As shown, adhesive patch 326 may feature an aperture 398 aligned with aperture 396 such that sensor 338 may pass through a bottom of on-skin sensor assembly 360 and through adhesive patch 326.

FIG. 3B illustrates a bottom perspective view of on-skin sensor assembly 360 of FIG. 3A. FIG. 3B further illustrates aperture 396 disposed substantially in a center portion of a bottom of on-skin sensor assembly 360, and aperture 398, both adapted for sensor 338 and needle insertion.

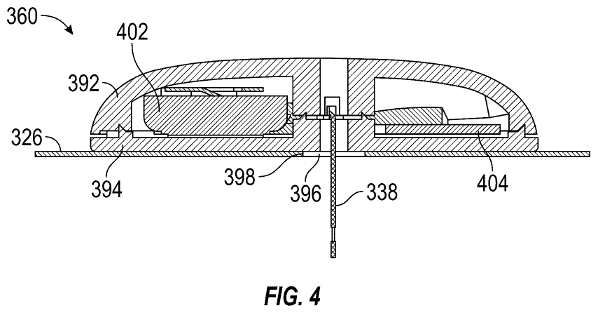

FIG. 4 illustrates a cross-sectional view of on-skin sensor assembly 360 of FIGS. 3A and 3B. FIG. 4 illustrates first, top portion 392 and second, bottom portion 394 of the outer housing, adhesive patch 326, aperture 396 in the center portion of on-skin sensor assembly 360, aperture 398 in the center portion of adhesive patch 326, and sensor 338 passing through aperture 396. The electronics unit, previously described in connection with FIG. 3A, may further include circuit board 404 and battery 402 configured to provide power to at least circuit board 404.

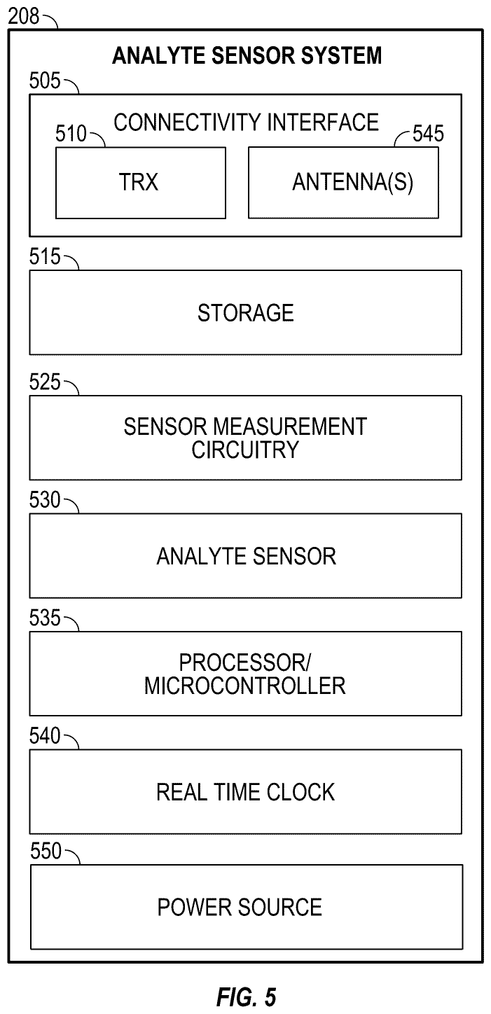

Turning now to FIG. 5, a more detailed functional block diagram of analyte sensor system 208 (discussed above, for example, in connection with FIGS. 1 and 2) is provided. As noted above, the analyte sensor system 208 may be an example of the analyte sensor system 8 illustrated in FIG. 1. As shown in FIG. 5, analyte sensor system 208 may include an analyte sensor 530 (e.g., which may be an example of the analyte sensor 10 illustrated in FIG. 1) coupled to sensor measurement circuitry 525 for receiving, processing, and managing sensor measurement values, obtained from the analyte sensor 530, indicative of analyte levels of a user of the analyte sensor system 208. Sensor measurement circuitry 525 may be coupled to processor/microcontroller 535. In some embodiments, processor/microcontroller 535 may include one or more processors and may be part of analyte sensor electronics module 12 in FIG. 1. In some embodiments, processor/microcontroller 535 may perform part or all of the functions of sensor measurement circuitry 525 for obtaining and processing sensor measurement values from the analyte sensor 530 and generating analyte data representative of the sensor measurement values. In some embodiments, the processed analyte data may be stored in storage 515, including one or more memories.

Processor/microcontroller 535 may be further coupled to a radio unit or transceiver 510 (e.g., which may be part of analyte sensor electronics module 12 in FIG. 1). In some embodiments, the processor/microcontroller 535 may be configured to provide sending sensor data, such as the analyte data, and other data to the transceiver 510 for transmission to an external device, such as display device 210 (referencing FIG. 2 by way of example). The transceiver 510 may also be configured to receive, from the external device, control information including requests for certain information and commands to perform certain actions. In some cases, the transceiver 510 may include logic or circuitry for communicating (e.g., transmitting and receiving) using different communication protocols, such as near-field communication (NFC), WiFi, Third Generation Partnership Project (3GPP)-based wireless communication protocols, short range communication protocols, such as Bluetooth, Bluetooth Low Energy (BLE), or other wireless communication protocols.

In some embodiments, the transceiver 510 may be coupled to an antenna system 545 associated with the connectivity interface 505, allowing the analyte sensor system 208 to wirelessly transmit and receive data. For example, the transceiver 510 may be configured to output data, such as the analyte data for wireless transmission via at least one antenna of the antenna system 545 or may be configured to obtain data that is wirelessly received via at least one of the antennas of the antenna system 545. In some cases, the antenna system 545 may be tuned to a particular frequency depending on a communication protocol used for communicating data. For example, in some embodiments, the antenna system 545 may include one or more antennas tuned for communicating data via a BLE protocol (e.g., tuned to 2.4 gigahertz). In some embodiments, the antenna system 545 may include one or more antennas tuned for communicating data via an NFC protocol (e.g., tuned to 13.56 megahertz).

Analyte sensor system 208, in example implementations, gathers analyte data using the analyte sensor 530 and transmits the same or a derivative thereof to display device 310, partner device 315, and/or server system 334 using the transceiver 510 and antenna system 545. Data points regarding analyte values may be gathered and transmitted over the life of the analyte sensor 530. New measurements and/or related information may be transmitted often enough for a remote device/individual to adequately monitor analyte (e.g., glucose) levels.

It is to be appreciated that some details of the processing, gathering, and exchanging data by analyte sensor system 208, partner devices 315, and/or display device 310 etc. are provided elsewhere herein. It will be appreciated upon studying the present disclosure that analyte sensor system 208 may contain several like components that are described with respect to FIG. 1 or 2, at least for some embodiments herein. The details and uses of such like components may therefore be understood vis-a-vis analyte sensor system 208 even if not expressly described here with reference to FIG. 5.

### Aspects Related to Short-Range Wireless Connection Strength Indication in an Analyte Monitoring System

As noted above, analyte sensor system 208 and the display device 210 of FIG. 2 may communicate with each other using various wireless communication protocols. In some embodiments, the analyte sensor system 208 may communicate using a short-range wireless connection, such as a Bluetooth connection (e.g., Bluetooth classic, Bluetooth Low Energy, etc.). Bluetooth is a short-range wireless technology that uses radio waves to transmit data between devices, such as the analyte data and the control information exchanged between the analyte sensor system 208 and the display device 210, described above. Bluetooth devices, such as the analyte sensor system 208 and display device 210, may rely on a stable and strong signal to communicate data in one or more Bluetooth transmissions accurately and consistently. If a signal strength associated with these Bluetooth transmissions is weak or intermittent, this may lead to data packets being lost or corrupted. Lost or corrupted data packets may result in several negative effects. For example, lost or corrupted packets may lead to a user of the analyte sensor system 208 being unable to check their analyte levels, which may be dangerous in certain circumstances (e.g., hypoglycemic or hyperglycemic events). Further, lost or corrupted packets may lead to additional retransmissions of these packets, which consumes additional power and is problematic in fixed-power devices, such as the analyte sensor system 208.

The signal strength associated with Bluetooth transmissions between the analyte sensor system 208 and display device 210 may be negatively affected by various factors. For example, in some cases, the signal strength may be negatively affected based on interference from other Bluetooth devices in a same area as the analyte sensor system 208 and/or display device 210. In some cases, the signal strength may be negatively affected based on there being a large distance between the analyte sensor system 208 and display device 210, such as when the user of the analyte sensor system 208 sets down the display device 210 and walks a significant distance away from the display device 210. In some cases, signal strength may be negatively affected based on the user being in a certain position (e.g., laying down on the analyte sensor system) or based on the user placing the display device 210 in a particular position or location (e.g., the user places the display device in their back pocket). In some cases, signal strength may be negatively affected based on obstructions between the analyte sensor system 208 and the display device 210.

As can be seen, there are numerous factors that may affect signal strength of communications between the analyte sensor system 208 and display device 210, each of which may result in lost packets and negative effects associated therewith. In general, a user of the analyte sensor system 208 may prefer to avoid scenarios that lead to a reduction in signal strength and resulting lost packets as these scenarios may otherwise lead to a decrease in battery life of the analyte sensor system 208 and/or the user not being provided their analyte levels. However, current analyte monitoring systems (e.g., including the analyte sensor system 208 and associated display device 210) may not provide, to the user, an indication of the signal strength of Bluetooth transmissions between the analyte sensor system 208 and display device 210, preventing the user from knowing that Bluetooth communication between the analyte sensor system 208 and the display device 210 is degrading. Additionally, without the knowledge that the Bluetooth communication between the analyte sensor system 208 and the display device 210 is degrading, the user may not be afforded an opportunity to take preventative actions to avoid signal loss events and lost packets.

Accordingly, aspects of the present disclosure provide techniques to help avoid or reduce signal loss events associated with a short-range wireless connection, such as a Bluetooth connection (e.g., Bluetooth classic, BLE, etc.) or other short-range connections, between an analyte sensor system and display device. In some embodiments, these techniques may include providing, to the user of the analyte sensor system, an indication of a signal strength of a short-range wireless connection between the analyte sensor system and the display device, allowing the user to take preventative action to avoid or reduce these signal loss events. As will be described below in greater detail, in some embodiments, the indication of the signal strength may comprise a signal strength indicator displayed in an application of the display device (e.g., a number of bars). In some embodiments, the indication of the signal strength may include a notification of a signal strength event (e.g., a signal strength falling below a threshold). In some embodiments, these notifications may be based on predictions of an expected signal strength event. In some embodiments, the indication of the signal strength may comprise an auditory alert or tone. In some embodiments, the notification of the signal strength event may be relayed by the display device to a smart watch of the user.

The techniques for avoiding or reducing signal loss events may also include corrective actions, such as automatically increasing a transmission power. Increasing the transmission power may increase a transmission range between the analyte sensor system and the display device, avoiding or reducing signal loss events and lost packets. In some embodiments, the increase in transmission power may be negotiated between the analyte sensor system and the display device. In some embodiments, the increase in transmission power may be based on a predicted signal strength event.

By avoiding or reducing these signal loss events, a connection between the analyte sensor system and thee display device may be maintained without interruption, allowing the user to be continuously informed regarding their analyte levels. Additionally, avoiding or reducing these signal loss events may reduce a number of lost or corrupted packets, thereby reducing a number of retransmissions of packets between the analyte sensor system and display device, and reducing power consumption.

### Example Operations of Devices in an Analyte Monitoring System

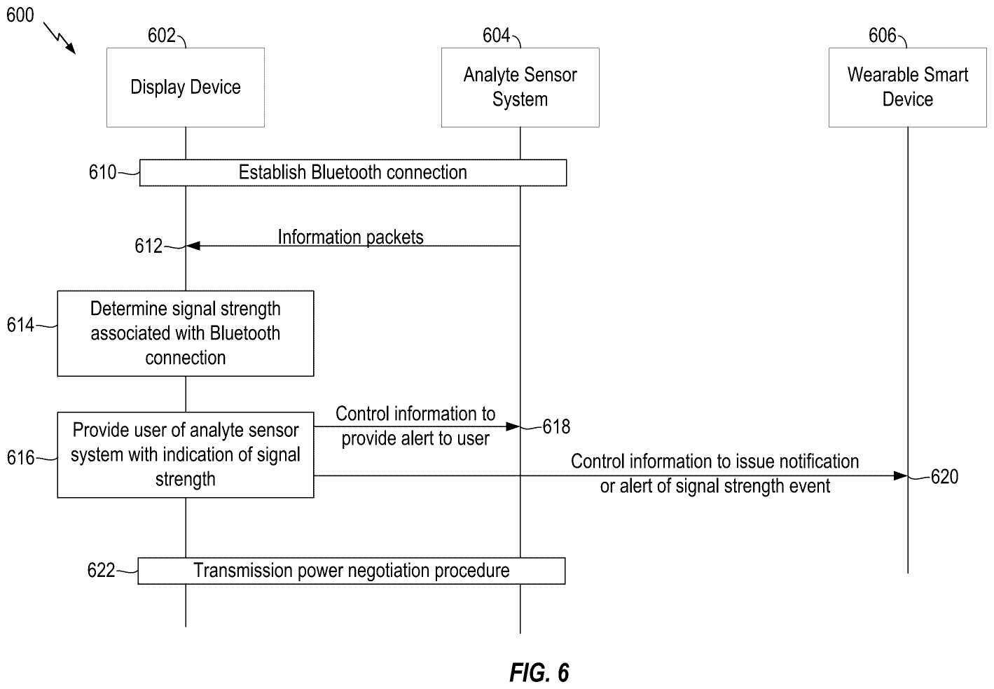

FIG. 6 depicts a process flow including operations 600 for communications in an analyte monitoring system between a display device 602 and an analyte sensor system 604. In some aspects, the operations 600 may be performed by the display device 602 and analyte sensor system 604 to avoid or reduce signal loss events associated with a short-range wireless connection between the display device 602 and the analyte sensor system 604 in the analyte monitoring system. In some aspects, the display device 602 may be an example of the display devices 110, 120, 130, 140 depicted and described with respect to FIG. 1 and/or the display device 210 depicted and described with respect to FIG. 2. For example, in some embodiments, the display device 602 may be a smartphone, a smart watch, or a dedicated receiver device for receiving and displaying analyte data to a user of the analyte sensor system 604. Similarly, the analyte sensor system 604 may be an example of analyte sensor system 8 depicted and described with respect to FIG. 1 and/or the analyte sensor system 208 depicted and described with respect to FIG. 2.

As shown, operations 600 begin at 610 with the display device 602 establishing a short-range wireless connection with the analyte sensor system 604. In some embodiments, the short-range wireless connection may comprise a Bluetooth connection, a Bluetooth Low Energy (BLE) connection, or other short-range wireless connections as described herein.

At 612, the display device 602 receives one or more information packets from the analyte sensor system 604. In some embodiments, the information packets may comprise sensor information, such as estimated glucose value (EGV) packets, including analyte data indicating analyte levels of the user of the analyte sensor system 604. In some embodiments, the information packets may include other information, such as a reference signal.

At 614, the display device 602 determines a signal strength associated with the short-range wireless connection based on the one or more information packets received from the analyte sensor system 604. In some embodiments, the display device 602 may determine the signal strength based on received signal strength indicator (RSSI) measurements. For example, in some embodiments, determining the signal strength at 614 may include performing RSSI measurements on the one or more information packets received from the analyte sensor system 604.

In some embodiments, determining the signal strength associated with the short-range wireless connection at 614 may include determining a bit error rate (BER) associated with the one or more information packets received from the analyte sensor system. For example, the display device 602 may determine that the signal strength is a first value (e.g., associated with a weak short-range wireless connection) based on the BER associated with one or more information packets received from the analyte sensor system 604 being above a threshold. In other embodiments, the display device 602 may determine that the signal strength is one or more second values (e.g., associated with a stronger short-range wireless connection) based on the BER associated with one or more information packets received from the analyte sensor system 604 being less than or equal to the threshold. In general, the signal strength associated with the short-range wireless connection may be different for different BERs associated with the one or more information packets received from the analyte sensor system 604.

In some embodiments, the analyte sensor system 604 may be configured to perform RSSI or BER measurements based on signals received from the display device 602. For example, in some embodiments, the display device 602 may transmit one or more signals to the analyte sensor system 604. The analyte sensor system 604 may then be configured to perform RSSI measurements and/or BER measurements on the one or more signals received from the display device 602. Thereafter, the analyte sensor system 604 may then transmit the RSSI measurements and/or BER measurements to the display device 602. The display device 602 may then determine the signal strength associated with the short-range wireless connection at 614 based on the RSSI measurements and/or BER measurements received from the analyte sensor system.

In some embodiments, the display device 602 may determine the signal strength associated with the short-range wireless connection at 614 by determining a number of the one or more information packets received from the analyte sensor system in a period of time. For example, the display device 602 may determine that the signal strength is a first value (e.g., associated with a weak short-range wireless connection) based on the number of the one or more information packets received from the analyte sensor system 604 dropping below a threshold number of packets in the period of time. In other embodiments, the display device 602 may determine that the signal strength is one or more second values (e.g., associated with a stronger short-range wireless connection) based on the number of the one or more information packets received from the analyte sensor system 604 being greater than or equal to the threshold number of packets in the period of time. In general, the signal strength may be different for different numbers of the one or more information packets received from the analyte sensor system 604 in the period of time.

As shown at 616, operations 600 further include the display device 602 providing, to the user of the analyte sensor system 604, an indication of the determined signal strength associated with the short-range wireless connection. As will be described in greater detail below, providing the user with the indication of the determined signal strength may help the user take preventative actions to avoid a signal loss event associated with the short-range wireless connection and improve the signal strength.

In some embodiments, the signal strength indication comprises the RSSI measurements of the one or more information packets. In some embodiments, the signal strength indication may be based on the BER associated with the one or more information packets received from the analyte sensor system 604. In some embodiments, the signal strength indication is based on the number of the one or more information packets received from the analyte sensor system 604 in the period of time.

In some embodiments, the indication of the signal strength may be provided to the user of the analyte sensor system 604 in different manners. For example, in some embodiments, providing the indication of the signal strength at 616 may comprise displaying, on a display of the display device 602, a signal strength indictor represented by number of shapes that corresponds to the signal strength. The shapes may comprise at least one of bars, a circles, of squares, or other shapes for representing the signal strength. In some embodiments, different numbers of the shapes correspond to different signal strengths.

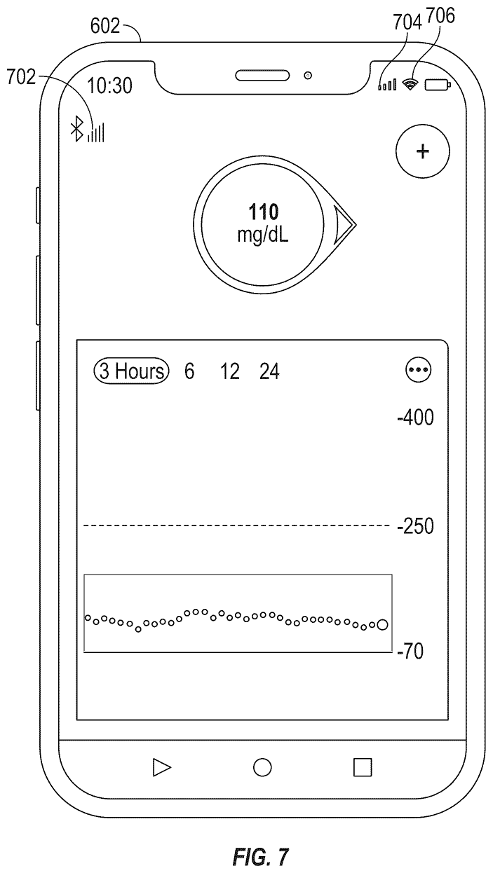

FIG. 7 illustrates an example of providing an indication of the signal strength of the short-range wireless connection as a signal strength indicator represented as a number of bars. For example, as shown in FIG. 7, the display device 602 may be an example of a smartphone that is executing an analyte monitoring application for monitoring analyte levels of a user of the analyte sensor system 604. While the display device 602 is illustrated as a smartphone in FIG. 7, it should be understood that the display device 602 may be any other device capable of displaying analyte data received from the analyte sensor system, such as a dedicated receiver device.

As shown, the display device 602 may provide the indication of the signal strength associated with the short-range wireless connection as a signal strength indicator 702 represented as a number of bars. Further, as shown, the signal strength indicator 702 indicating the signal strength associated with the short range connection may be provided in addition to other signal strength indicators associated with other wireless connections of the display device 602, such as a cellular signal strength indicator 704 and a WiFi signal strength indicator 706. While the signal strength indicator 702 is represented as a number of bars in FIG. 7, it should be understood that other shapes may also be used (e.g., dots, dashes, circles, squares, or the like). Accordingly in this embodiment, each different number of bars displayed by the display device 602 may correspond to a different signal strength associated with the short-range wireless connection between the display device 602 and the analyte sensor system 604. For example, in some embodiments, a higher number of bars may be displayed for higher signal strength (e.g., RSSI) values whereas a lower number of bars may be displayed for lower signal strength values.

As an example, the number of bars or shapes of the signal strength indicator 702 displayed on the display of the display device 602 may comprise a first number of bars when the signal strength associated with the short-range wireless connection is below a first threshold. Additionally, the number of bars or shapes of the signal strength indicator 702 displayed on the display comprises a second number of bars or shapes, greater than the first number of bars or shapes, when the signal strength associated with the short-range wireless connection is greater than or equal to the first threshold and less than a second threshold. Additionally, the number of bars or shapes of the signal strength indicator 702 displayed on the display may comprise a third number of shapes, greater than the second number of bars or shapes, when the signal strength associated with the short-range wireless connection is greater than or equal to the second threshold, and so on. In some cases, a lower number of bars of the signal strength indicator 702, indicating a lower signal strength, may prompt the user to take corrective action to improve the signal strength of the short-range wireless connection between the analyte sensor system 604 and display device 602, such as repositioning the display device 602, leaving an area with high interference or blockages, etc.

In some cases, the user of the analyte sensor system 604 may set the display device 602 down and walk away from it or may place the display device somewhere (e.g., a back pocket, etc.) that results in poor signal strength, preventing the user from being able to actively monitor the signal strength indictor displayed on the display device 602. To address these scenarios, in some embodiments, the indication of the signal strength associated with the short-range wireless connection may comprise a notification of a signal strength event associated with the short-range wireless connection determined, for example, in step 614 of FIG. 6. For example, in some embodiments, the display device 602 may determine the signal strength event based on an RSSI associated with the one or more information packets received from the analyte sensor system being below a signal strength threshold. In some embodiments, the display device 602 may determine the signal strength event based on a BER associated with the one or more information packets received from the analyte sensor system being greater than or equal to a BER threshold. In some embodiments, the display device 602 may determine the signal strength event based on a number of the one or more information packets (e.g., EGV packets) being below a threshold number for a period of time.

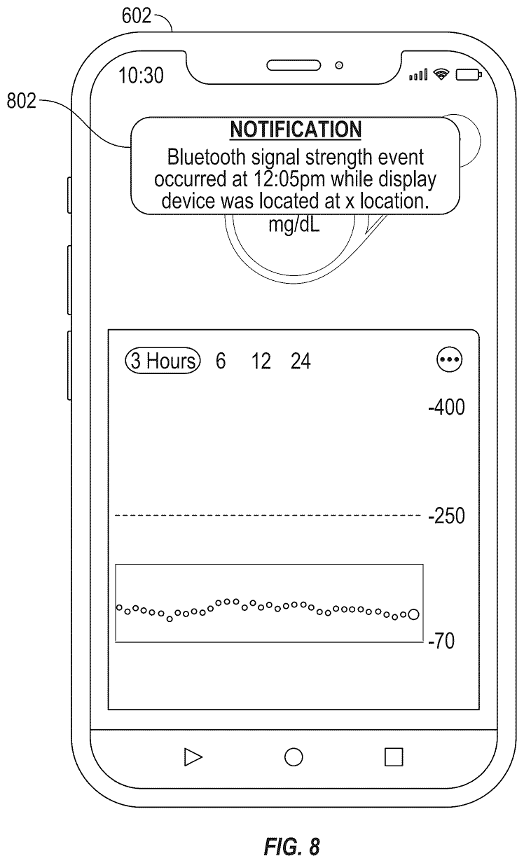

FIG. 8 illustrates an example of providing an indication of the signal strength of the short-range wireless connection as a notification of a signal strength event. For example, as shown in FIG. 8, the display device 602 may be an example of a smartphone that is executing an analyte monitoring application for monitoring analyte levels of a user of the analyte sensor system 604. While the display device 602 is illustrated as a smartphone in FIG. 8, it should be understood that the display device 602 may be any other device capable of displaying analyte data received from the analyte sensor system, such as a dedicated receiver device.

As shown, the display device 602 may provide the indication of the signal strength associated with the short-range wireless connection as a notification 802 of a signal strength event. Additionally, as shown, the notification 802 may include a time period indicating when the signal strength event occurred (e.g., 12:05 pm). Additionally, in some embodiments, the notification 802 may include location information of the display device 602 (e.g., x location) indicating where the display device 602 was located when the signal strength event occurred. In some embodiments, the display device 602 may determine or record its location based on, for example, global positioning system (GPS) data, accelerometer data, and the like. In some embodiments, based on the notification of the signal strength event and associated timing and/or location information, the user of the analyte sensor system 604 may be able to take future corrective actions to avoid or reduce these signal strength events, such as carrying the display device 602 with them, repositioning the display device 302 (e.g., placing the display device in a front pocket, etc.), leaving an area with high wireless interference, or similar actions corrective actions to avoid or reduce these signal strength events.

In other words, in some embodiments, the notification 802 may prompt the user to take corrective action to avoid or reduce the occurrence of future signal strength events or to improve the signal strength of the short-range wireless connection. In some embodiments, the notification 802 may include an indication of a corrective action for the user to take. In some embodiments, the indication of the corrective action comprises at least one of an indication for the user to reposition the display device 602 or an indication for the user to leave an area having an interference level, caused by transmissions associated with other devices, above a threshold.

More specifically, for example, in some embodiments, the notification 802 may provide an indication of corrective actions that the user may take to improve the signal strength of the short-range wireless connection. For example, in some embodiments, based on RSSI measurements, the display device 602 may determine that an obstruction between the analyte sensor system 604 and the display device 602 has occurred, there is too much interference in a present location of the user, or the like. In such embodiments, the display device 602 may include an indication of this obstruction within the notification 802 displayed to the user, and may instruct the user to reposition the display device 602 or the analyte sensor system 604, or may instruct the user to leave the area with high interference.

In some embodiments, upon triggering of a signal strength event, the display device 602 may be configured to wait for a threshold amount of time before providing the notification 802 to the user of the signal strength event. For example, in some embodiments, this threshold amount of time may be used by the display device 602 to determine whether the signal strength associated with the short-range wireless connection increases again above the signal strength threshold. For example, in some embodiments, the user may set the display device 602 down and walk to another room, which may cause the signal strength associated with the short-range wireless connection between the analyte sensor system 604 and display device 602 to drop below signal strength threshold. If the user returns to the display device 602 within the threshold amount of time, causing the signal strength to increase above the signal strength threshold, the display device 602 may be configured to refrain from providing the notification 802 to the user indicating the signal strength event.

Accordingly, in some embodiments, providing the indication of the determined signal strength associated with the short-range wireless connection at 616 in FIG. 6 may include the display device 602 determining that the signal strength associated with the short-range wireless connection is below the threshold signal strength and providing the notification 802 of the signal strength event to the user when the signal strength is below the threshold signal strength for the threshold amount of time. In some embodiments, the threshold amount of time may be configurable by the user of the display device.

In some embodiments, the notification 802 of the signal strength event provided to the user at 616 in FIG. 6 may comprise a notification of a signal strength event predicted to occur in the future, which may be based on a plurality of signal strength events that have occurred in the past. In other words, the signal strength event predicted to occur in the future may be based on a pattern of previous signal strength events. In some embodiments, determining the signal strength event predicted to occur in the future based on the plurality of signal strength events that have occurred in the past may include determining that the signal strength events in the plurality of signal strength events that have occurred in the past have occurred (1) at approximately the same time each day, (2) at approximately regular intervals, and/or (3) in a same approximate location.

For example, in some cases, when getting ready to sleep, the user of the analyte sensor system 604 may routinely place the display device 602 down in a location that leads to signal strength events at a particular time each night. In this example, the display device 602 may determine that this signal strength event usually occurs at the particular time each night and may provide a predictive notification to the user prior to this particular time at night. The predictive notification may indicate to the user that a signal strength event is expected to occur at the particular time and may request that the user take corrective action to avoid the signal strength event.

In some embodiments, the indication of the signal strength provided to the user at 616 in FIG. 6 may comprise an alert of a signal strength event. For example, in some embodiments, the alert may include at least one of an auditory alert, a vibratory alert, or a light emitting diode (LED) alert. In some embodiments, providing the indication of the determined signal strength may include providing the alert to the user when the determined signal strength reaches a certain signal strength threshold. In some embodiments, the certain signal strength threshold a minimum signal strength threshold in which communication between the analyte sensor system 604 and the display device 602 is still possible on the short-range wireless connection

For example, in some embodiments, the display device 602 may monitor the signal strength of the short-range wireless connection between the analyte sensor system 604 and the display device 602 (e.g., in steps 612 and 614 of FIG. 6). When the signal strength drops below the certain threshold, the display device 602 may provide the alert. The alert may signify to the user that the signal strength of the short-range wireless connection between the analyte sensor system 604 and the display device 602 is diminishing and to take corrective action to improve the signal strength. As such, providing the alert to the user may allow the user to take corrective action before the short-range wireless connection between the analyte sensor system 604 and the display device 602 is lost and communication on the short-range wireless connections is no longer possible.

In some embodiments, in step 614 of FIG. 6, the display device 602 may determine that the signal strength associated with the short-range wireless connection has dropped below the threshold, indicating a signal strength event has occurred. In this case, when providing the indication of the signal strength associated with the short-range wireless connection, the display device 602 may transmit control information to the analyte sensor system 604, instructing the analyte sensor system 604 to provide the alert to the user, as shown at 618 in FIG. 6. For example, the control information may instruct the analyte sensor system 604 to provide an auditory alert or tone (e.g., using a built-in speaker), a haptic-based alert (e.g., using a vibratory device), an LED-based alert using an LED indicator light, or the like to signify to the user that the signal strength of the short-range wireless connection is weak or diminishing. In some embodiments, the display device 602 may be configured to provide the user with an option to silence the alerts (e.g., issued based on the signal strength of the short-range wireless connection) for a period of time to avoid annoyance of the user.

In some embodiments, the control information instructing the analyte sensor system 604 to provide the alert to the user may prompt the user to look at the display of the display device 602. In some embodiments, after providing the alert to the user, the display device 602 may also provide, on the display of the display device 602, a notification 802 of the signal strength event, including additional information associated with the signal strength event, as described with respect to FIG. 8. For example, in some cases, the additional information may include at least one of: a time period associated with the signal strength event, location information indicating a location of the display device when the signal strength event occurred, one or more corrective actions for the user to take to avoid future signal strength events or to avoid loss of the short-range wireless connection.

In some embodiments, as noted above, the display device 602 may be a smart phone. Further, in some cases, the user may also be wearing a smart device, such as a wearable smart device 606 illustrated in FIG. 6, which is paired with the smart phone. In some embodiments, the wearable smart device 606 may be an example of at least one a smart watch, smart glasses, or a virtual reality headset. In such embodiments, when the display device 602 determines that a signal strength event has or will occur (e.g., based on the RSSI associated with the short-range wireless connection between the analyte sensor system 604 and display device 602 dropping below a threshold, BER being greater than a threshold, the number of the one or more information packets being below a threshold for a period of time, etc.), providing the indication of the signal strength in step 616 of FIG. 6 may include the display device 602 transmitting control information to the wearable smart device 606 instructing the wearable smart device 606 to issue a notification or alert to the user of the signal strength event, as shown in step 620 of FIG. 6. This may be helpful in scenarios in which the user sets the display device 602 down and walks away from it while still wearing the wearable smart device 606. For example, in such embodiments, while the user may not be able to see notifications provided locally by the display device 602, the user may still be notified of the signal strength event by the display device 602 via the wearable smart device 606.

In some embodiments, the display device 602 and analyte sensor system 604 may negotiate an increased transmission power that may help to avoid the short-range wireless connection from being lost. For example, in some embodiments, the display device 602 may determine one or more signal strength events associated with the short-range wireless connection based on the signal strength determined in step 614 of FIG. 6 being less than or equal to a certain signal strength threshold. In some embodiments, the certain threshold may comprise a minimum signal strength threshold that communications on the short-range wireless connection may still be properly transmitted and received (or some other signal strength value above the minimum). Thereafter, as illustrated at 622 in FIG. 6, in response to determining the one or more signal strength events, the display device 602 may perform a transmission power negotiation procedure with the analyte sensor system 604.

In some embodiments, performing the transmission power negotiation procedure at 622 in FIG. 6 may include the display device 602 transmitting message to the analyte sensor system 604 for increasing a transmission power for transmitting the one or more information packets. For example, when a signal strength event is determined by the display device 602, the display device 602 may transmit the message to the analyte sensor system 604 to indicate to the analyte sensor system 604 to increase a transmission power of the one or more information packets transmitted by the analyte sensor system 604 to the display device 602. Increasing the transmission power of the one or more information packets may increase the communication range between the display device 602 and analyte sensor system 604, allowing the short-range wireless connection to be maintained even after a signal strength event occurs (e.g., as a result of a previous, lower transmission power). For example, if the user of the analyte sensor system 604 sets the display device 602 down and walks away, at a certain point the signal strength associated with the short-range wireless connection may drop below the certain threshold. When this occurs, the display device 602 may instruct the analyte sensor system 604 to increase the transmission power of the one or more information packets so that the short-range wireless connection may be maintained even though the user of the analyte sensor system 604 is located at a significant distance away from the display device 602.

In some embodiments, the message transmitted by the display device 602 to the analyte sensor system for increasing a transmission power for transmitting the one or more information packets may indicate a specific value for the transmission power for the analyte sensor system 604 to use for transmitting the one or more information packets. In some embodiments, the message may indicate the transmission power for the one or more information packets as an offset from a current transmission power of the one or more information packets. In some embodiments, the offset may be applied by the analyte sensor system 604 on top of the current transmission power.

In some embodiments, the message for increasing a transmission power for transmitting the one or more information packets may request the analyte sensor system 604 to transmit a power headroom report to the display device 602 that indicates an amount of transmission power that the analyte sensor system 604 is capable of increasing to. Thereafter, the display device 602 may receive the power headroom report from the analyte sensor system 604 based on the transmitted request. The display device 602 may then determine the transmission power for the analyte sensor system 604 to use for transmitting the one or more information packets based on the power headroom report. The display device 602 may then transmit, to the analyte sensor system 604, an indication of the determined transmission power for the analyte sensor system 604 to use for transmitting the one or more information packets. In some embodiments, the display device 602 may receive an acknowledgement message from the analyte sensor system 604, acknowledging that the analyte sensor system 604 will increase its transmission power based on the indication of the determined transmission power transmitted to the analyte sensor system.

In some embodiments, the analyte sensor system 604 may be configured to initiate the transmission power negotiation based on determination that the signal strength of transmissions received from the display device 602 is less than or equal to the threshold. For example, based on this determination, the analyte sensor system 604 may transmit a message to the display device 602 indicating that the analyte sensor system 604 will be increasing the transmission power of the one or more information packets and requesting that the display device 602 also increase a transmission power used for transmitting information packets to the analyte sensor system 604.

In some embodiments, the message transmitted by the analyte sensor system 604 to the display device 602 indicating that the analyte sensor system 604 will be increasing the transmission power of the one or more information packets may indicate the transmission power that the analyte sensor system 604 will use to transmit the one or more information, for example, as an explicit indication of the transmission power or as a power offset from a current transmission power used by the analyte sensor system 604 to transmit the one or more information packets. In some embodiments, the message transmitted by the analyte sensor system 604 to the display device 602 indicating that the analyte sensor system 604 will be increasing the transmission power of the one or more information packets may indicate the transmission power for the display device 602 to use when transmitting signals to the analyte sensor system, for example, as an explicit indication of the transmission power or as a power offset from a current transmission power used by the display device 602.

As noted above, the display device 602 may be configured to predict that the one or more signal strength events will occur at a particular time in the future based on one or more past signal strength events. In some embodiments, performing the transmission power negotiation procedure with the analyte sensor system 604 in step 622 of FIG. 6 may include performing the transmission power negotiation procedure with the analyte sensor system 604 prior to the signal strength event occurring at the particular time in the future. In other words, in response to determining that a signal strength event is predicted to occur at the particular time, the display device 602 may be configured to automatically initiate the transmission power negotiation with the analyte sensor system 604 prior to the predicted signal strength event. For example, in response to determining that a signal strength event is predicted to occur at the particular time, the display device 602 may preemptively instruct the analyte sensor system 604 to increase the transmission power of the one or more information packets to avoid the signal strength event actually occurring, thereby avoiding issues with the short-range wireless connection (e.g., lost packets, retransmissions, etc.) between the display device 602 and analyte sensor system 604.

In some embodiments, the display device 602 may automatically initiate the transmission power negotiation procedure with the analyte sensor system 604 based on the predicted signal strength event when the predicted signal strength event continues to occur after notifying the user of the predicted signal strength event. For example, in some embodiments, the display device 602 may be configured provide a notification to the user of the predicted one or more signal strength events, as discussed above. Thereafter, if the display device 602 detects that the predicted one or more signal strength events have continued to occur a threshold number of times after providing the notification to the user of the predicted one or more signal strength events, the display device 602 may be configured to automatically initiate, at step 622 of FIG. 6, the transmission power negotiation procedure with the analyte sensor system prior to the next predicted signal strength event (e.g., may automatically instruct the analyte sensor system 604 to increase its transmission power). This automatic initiation of the transmission power negotiation procedure may help to conserve power resources as it provides the user with a certain number of instances in which to take corrective action to improve the signal strength between the analyte sensor system 604 and display device 602 before automatically increasing the transmission power for communications between the analyte sensor system 604 and display device 602.

In some embodiments, after completion of the transmission power negotiation procedure, the display device 602 may be configured to provide the user with an indication of at least one of that the transmission power negotiation procedure has been completed or that the transmission power for transmitting the one or more information packets has been increased.

### Example Operations of a Display Device

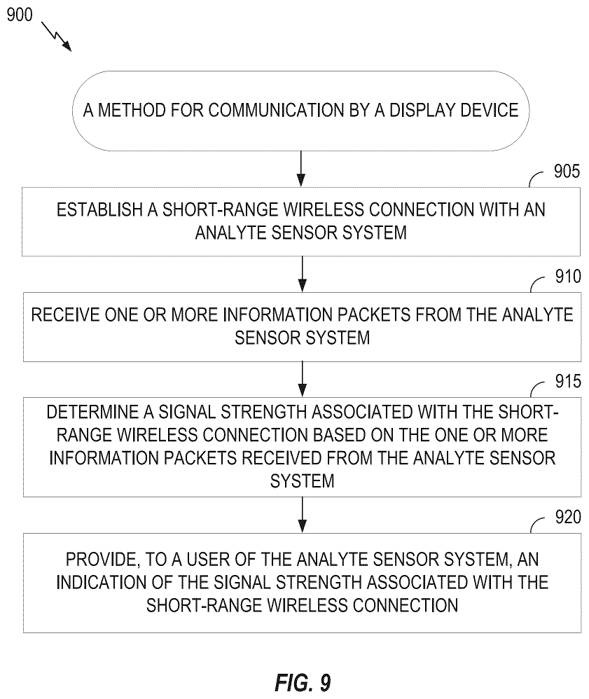

FIG. 9 shows an example of a method 900 of communication by a display device in an analyte monitoring system, such as at least one of the display devices 110, 120, 130, and 140 illustrated and described with respect to FIG. 1, the display device 210 illustrated and described with respect to FIG. 2, and/or the display device 602 illustrated and described with respect to FIG. 6.

Method 900 begins at step 905 with the display device establishing a short-range wireless connection with an analyte sensor system.

In step 910, the display device receives one or more information packets from the analyte sensor system.

In step 915, the display device determine a signal strength associated with the short-range wireless connection based on the one or more information packets received from the analyte sensor system.

In step 920, the display device provides, to a user of the analyte sensor system, an indication of the signal strength associated with the short-range wireless connection.

In some embodiments, determining the signal strength associated with the short-range wireless connection in step 915 comprises performing received signal strength indicator (RSSI) measurements on the one or more information packets received from the analyte sensor system. In some embodiments, the indication of the signal strength comprises the RSSI measurements of the one or more information packets.

In some embodiments, determining the signal strength associated with the short-range wireless connection in step 915 comprises determining a bit error rate (BER) associated with the one or more information packets received from the analyte sensor system. In some embodiments, the indication of the signal strength is based on the BER associated with the one or more information packets received from the analyte sensor system. In some embodiments, the indication of the signal strength is different for different BERs associated with the one or more information packets received from the analyte sensor system.

In some embodiments, determining the signal strength associated with the short-range wireless connection in step 915 comprises determining a number of the one or more information packets received from the analyte sensor system in a period of time. In some embodiments, the indication of the signal strength is based on the number of the one or more information packets received from the analyte sensor system in the period of time. In some embodiments, the indication of the signal strength is different for different numbers of the one or more information packets received from the analyte sensor system in the period of time.

In some embodiments, providing the indication of the signal strength in step 920 comprises displaying, on a display of the display device, a number of shapes that corresponds to the signal strength. In some embodiments, the shapes comprise at least one of bars, a circles, of squares, or other shapes for representing the signal strength. In some embodiments, different numbers of the shapes correspond to different signal strengths.

In some embodiments, the number of shapes displayed on the display comprises a first number of shapes when the signal strength associated with the short-range wireless connection is below a first threshold. In some embodiments, the number of shapes displayed on the display comprises a second number of shapes, greater than the first number of shapes, when the signal strength associated with the short-range wireless connection is greater than or equal to the first threshold and less than a second threshold. In some embodiments, the number of shapes displayed on the display comprises a third number of shapes, greater than the second number of shapes, when the signal strength associated with the short-range wireless connection is greater than or equal to the second threshold.

In some embodiments, the indication of the signal strength associated with the short-range wireless connection comprises a notification of a signal strength event.

In some embodiments, the signal strength event occurs as a result of at least one of: a received signal strength indicator (RSSI) associated with the one or more information packets received from the analyte sensor system being below a signal strength threshold; a bit error rate (BER) associated with the one or more information packets received from the analyte sensor system being greater than or equal to a BER threshold; or a number of the one or more information packets being below a threshold number for a period of time.

In some embodiments, the notification includes a time period indicating when the signal strength event occurred.

In some embodiments, the notification includes location information of the display device indicating where the display device was located when the signal strength event occurred.

In some embodiments, the notification prompts the user to take corrective action to avoid or reduce occurrence of future signal strength events.

In some embodiments, the notification includes an indication of a corrective action for the user to take. In some embodiments, the indication of the corrective action comprises at least one of: an indication for the user to reposition the display device; or an indication for the user to leave an area having an interference level, caused by transmissions associated with other devices, above a threshold.

In some embodiments, providing the indication of the signal strength associated with the short-range wireless connection in step 920 comprises: determining that the signal strength associated with the short-range wireless connection is below a threshold signal strength; and providing the notification of the signal strength event to the user when the signal strength is below the threshold signal strength for a threshold amount of time. In some embodiments, the threshold amount of time is configurable by the user of the display device.

In some embodiments, the notification of the signal strength event comprises a notification of a signal strength event predicted to occur in the future. In some embodiments, the method 900 further includes determining the signal strength event predicted to occur in the future based on a plurality of signal strength events that have occurred in the past. In some embodiments, determining the signal strength event predicted to occur in the future based on the plurality of signal strength events that have occurred in the past comprises determining that the signal strength events in the plurality of signal strength events that have occurred in the past have occurred: at approximately a same time each day; at approximately regular intervals; or in a same approximate location.

In some embodiments, the indication of the signal strength associated with the short-range wireless connection comprises an alert of a signal strength event. In some embodiments, the alert includes at least one of an auditory alert, a vibratory alert, or a light emitting diode (LED) alert.

In some embodiments, providing the indication of the signal strength in step 920 comprises providing the alert to the user when the signal strength reaches a certain signal strength threshold. In some embodiments, the certain signal strength threshold a minimum signal strength threshold in which communication between the analyte sensor system and the display device is still possible on the short-range wireless connection. In some embodiments, providing the alert to the user when the signal strength reaches a certain signal strength threshold provides the user with an amount of time to take corrective action before communication between the analyte sensor system and the display device is no longer possible on the short-range wireless connection.

In some embodiments, the method 900 further includes transmitting control information to the analyte sensor system, instructing the analyte sensor system to provide the alert to the user. In some embodiments, the control information instructing the analyte sensor system to provide the alert to the user prompts the user to look at a display of the display device. In some embodiments, the method 900 further comprises providing, on the display of the display device, additional information associated with the signal strength event including at least one of: a time period associated with the signal strength event; location information indicating a location of the display device when the signal strength event occurred; or one or more corrective actions for the user to take to avoid future signal strength events or to avoid loss of the short-range wireless connection.

In some embodiments, the method 900 further comprises providing the user with an option to silence alarms of signal strength events for a period of time.

In some embodiments, providing the indication of the signal strength associated with the short-range wireless connection to the user in step 920 comprises transmitting the indication of the signal strength to one or more second display devices associated with the user.

In some embodiments, the one or more second display devices comprise at least one of a smart watch, smart glasses, or a virtual reality headset.

In some embodiments, the method 900 further includes determining one or more signal strength events associated with the short-range wireless connection based on the signal strength being less than or equal to a certain signal strength threshold. In some embodiments, the method 900 further comprises, in response to determining the one or more signal strength events, performing a transmission power negotiation procedure with the analyte sensor system.

In some embodiments, performing the transmission power negotiation procedure comprises transmitting message to the analyte sensor system for increasing a transmission power for transmitting the one or more information packets. In some embodiments, the message indicates a specific value for the transmission power for the analyte sensor system to use or indicates the transmission power as an offset from a current transmission power of the one or more information packets.

In some embodiments, the message requests the analyte sensor system to transmit a power headroom report to the display device indicating an amount of transmission power that the analyte sensor system is capable of increasing to. In some embodiments, the method 900 further comprises receiving the power headroom report from the analyte sensor system in response to the message.

In some embodiments, the method 900 further comprises determining the transmission power for the analyte sensor system to use for transmitting the one or more information packets based on the power headroom report. In some embodiments, the method 900 further comprises transmitting, to the analyte sensor system, an indication of the determined transmission power for the analyte sensor system to use for transmitting the one or more information packets.

In some embodiments, the method 900 further comprises receiving an acknowledgement message from the analyte sensor system acknowledging that the analyte sensor system will increase its transmission power based on the indication of the determined transmission power transmitted to the analyte sensor system.

In some embodiments, the method 900 further comprises predicting that the one or more signal strength events will occur at a particular time in the future based on one or more past signal strength events.

In some embodiments, performing the transmission power negotiation procedure with the analyte sensor system comprises performing the transmission power negotiation procedure with the analyte sensor system prior to the signal strength event occurring at the particular time in the future.

In some embodiments, providing the indication of the signal strength associated with the short-range wireless connection in step 920 comprises providing a notification to the user of the predicted one or more signal strength events.

In some embodiments, the method 900 further comprises detecting that the predicted one or more signal strength events have occurred a threshold number of times after providing the notification to the user of the predicted one or more signal strength events. In some embodiments, performing the transmission power negotiation procedure with the analyte sensor system comprises performing the transmission power negotiation procedure with the analyte sensor system after detecting that the predicted one or more signal strength events have occurred the threshold number of times.

In some embodiments, the method 900 further comprises, after completion of the transmission power negotiation procedure, providing the user with an indication of at least one of that the transmission power negotiation procedure has been completed or that the transmission power for transmitting the one or more information packets has been increased.

In some embodiments, the one or more information packets includes analyte data indicating analyte levels of the user of the analyte sensor system.

In some embodiments, the short-range wireless connection comprises a Bluetooth connection or a Bluetooth Low Energy (BLE) connection.

### Example Communications Device(s)

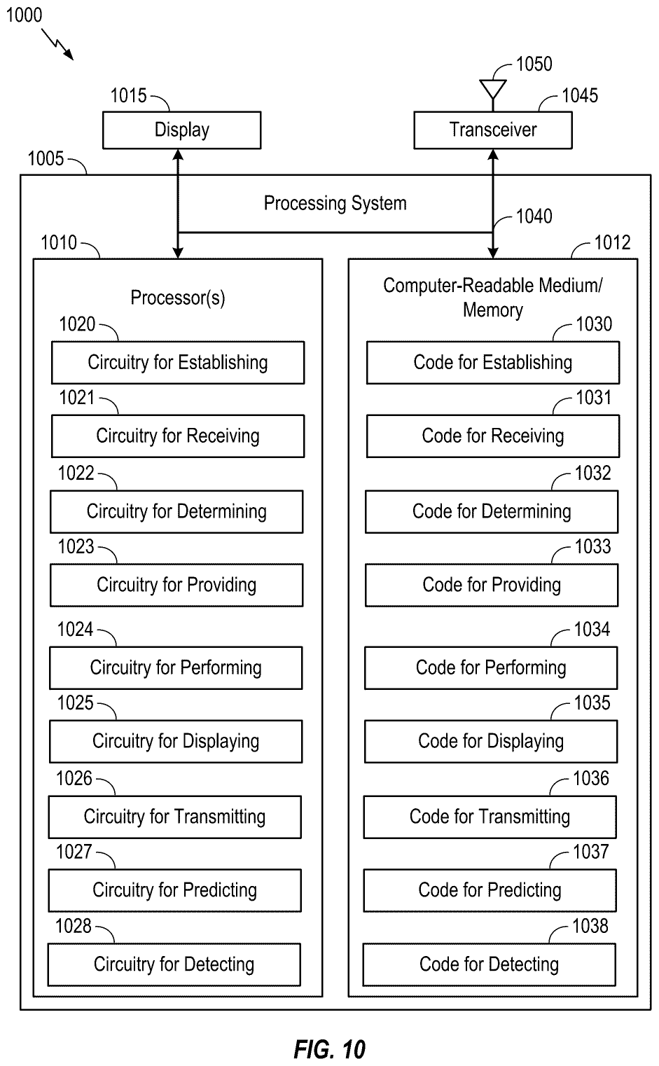

FIG. 10 depicts aspects of an example communications device 1000. In some aspects, communications device 1000 is a display device, such as at least one of the display devices 110, 120, 130, and 140 illustrated and described with respect to FIG. 1, the display device 210 illustrated and described with respect to FIG. 2, and/or the display device 602 illustrated and described with respect to FIG. 6.

As shown, the communications device 1000 includes a display 1015 for displaying sensor information and/or analyte data to a user and/or receiving inputs from the user. In some embodiments, the display 1015 may be configured to display additional information to the user, such as an indication of a signal strength associated with a short-range wireless connection, as described above. In some embodiments, the display 1015 may be representative touchscreen display 112, 122, 132,/or 142 illustrated and described with respect to FIG. 1.

The communications device 1000 further includes a processing system 1005 coupled to the transceiver 1045 (e.g., a transmitter and/or a receiver). The transceiver 1045 is configured to transmit and receive signals for the communications device 1000 via the antenna 1050, such as the various signals as described herein. The processing system 1005 may be configured to perform processing functions for the communications device 1000, including processing signals received (e.g., analyte data, user input, etc.) and/or to be transmitted by the communications device 1000.

The processing system 1005 includes one or more processors 1010. The one or more processors 1010 are coupled to a computer-readable medium/memory 1012 and the display 1015 via a bus 1040. In certain aspects, the computer-readable medium/memory 1012 is configured to store instructions (e.g., computer-executable code) that when executed by the one or more processors 1010, cause the communications device 1000 to perform one or more of the operations 600 described with respect to FIG. 6 and/or the method 900 described with respect to FIG. 9, or any aspect related to it. Note that reference to a processor performing a function of communications device 1000 may include one or more processors 1010 performing that function of communications device 1000.

In the depicted example, computer-readable medium/memory 1012 stores code (e.g., executable instructions), such as code for establishing 1030, code for receiving 1031, code for determining 1032, code for providing 1033, code for performing 1034, code for displaying 1035, code for transmitting 1036, code for predicting 1037, and code for detecting 1038. Processing of the code for establishing 1030, code for receiving 1031, code for determining 1032, code for providing 1033, code for performing 1034, code for displaying 1035, code for transmitting 1036, code for predicting 1037, and code for detecting 1038 may cause the communications device 1000 to perform one or more of the operations 600 described with respect to FIG. 6 and/or the method 900 described with respect to FIG. 9, or any aspect related to it.

The one or more processors 1010 include circuitry configured to implement (e.g., execute) the code stored in the computer-readable medium/memory 1012, including circuitry such as circuitry for establishing 1020, circuitry for receiving 1021, circuitry for determining 1022, circuitry for providing 1023, circuitry for performing 1024, circuitry for displaying 1025, circuitry for transmitting 1026, circuitry for predicting 1027, and circuitry for detecting 1028. Processing with circuitry for establishing 1020, circuitry for receiving 1021, circuitry for determining 1022, circuitry for providing 1023, circuitry for performing 1024, circuitry for displaying 1025, circuitry for transmitting 1026, circuitry for predicting 1027, and circuitry for detecting 1028 may cause the communications device 1000 to perform one or more of the operations 600 described with respect to FIG. 6 and/or the method 900 described with respect to FIG. 9, or any aspect related to it.

Various components of the communications device 1000 may provide means for performing one or more of the operations 600 described with respect to FIG. 6 and/or the method 900 described with respect to FIG. 9, or any aspect related to it. For example, means for transmitting, sending or outputting for transmission may include the transceiver 1045 and the antenna 1050 of the communications device 1000 in FIG. 10. Means for receiving or obtaining may include the transceiver 1045 and the antenna 1050 of the communications device 1000 in FIG. 10. Means for establishing, means for determining, means for providing, means for performing, means for predicting, and means for detecting may include one or more processors, such as the one or more processors 1010 of the communications device 1000 in FIG. 10. Means for displaying may include a display, such as the display 1015 of the communications device 1000 in FIG. 10, and/or one or more processors, such as the one or more processors 1010 of the communications device 1000 in FIG. 10.

### Example Clauses

Implementation examples are described in the following numbered clauses:

Clause 1: A method for communication by a display device in an analyte monitoring system, comprising: establishing a short-range wireless connection with an analyte sensor system; receiving one or more information packets from the analyte sensor system; determining a signal strength associated with the short-range wireless connection based on the one or more information packets received from the analyte sensor system; and providing, to a user of the analyte sensor system, an indication of the signal strength associated with the short-range wireless connection.

Clause 2: The method of Clause 1, wherein: determining the signal strength associated with the short-range wireless connection comprises performing received signal strength indicator (RSSI) measurements on the one or more information packets received from the analyte sensor system; and the indication of the signal strength comprises the RSSI measurements of the one or more information packets.

Clause 3: The method of any of Clauses 1-2, wherein: determining the signal strength associated with the short-range wireless connection comprises determining a bit error rate (BER) associated with the one or more information packets received from the analyte sensor system; and the indication of the signal strength is based on the BER associated with the one or more information packets received from the analyte sensor system.

Clause 4: The method of Clause 3, wherein the indication of the signal strength is different for different BERs associated with the one or more information packets received from the analyte sensor system.

Clause 5: The method of any of Clauses 1-4, wherein: determining the signal strength associated with the short-range wireless connection comprises determining a number of the one or more information packets received from the analyte sensor system in a period of time; and the indication of the signal strength is based on the number of the one or more information packets received from the analyte sensor system in the period of time.

Clause 6: The method of Clause 5, wherein the indication of the signal strength is different for different numbers of the one or more information packets received from the analyte sensor system in the period of time.

Clause 7: The method of any of Clauses 1-6, wherein providing the indication of the signal strength comprises displaying, on a display of the display device, a number of shapes that corresponds to the signal strength.

Clause 8: The method of Clause 7, wherein the shapes comprise at least one of bars, a circles, of squares, or other shapes for representing the signal strength.

Clause 9: The method of any of Clauses 7-8, wherein different numbers of the shapes correspond to different signal strengths.

Clause 10: The method of Clause 9, wherein: the number of shapes displayed on the display comprises a first number of shapes when the signal strength associated with the short-range wireless connection is below a first threshold; the number of shapes displayed on the display comprises a second number of shapes, greater than the first number of shapes, when the signal strength associated with the short-range wireless connection is greater than or equal to the first threshold and less than a second threshold; and the number of shapes displayed on the display comprises a third number of shapes, greater than the second number of shapes, when the signal strength associated with the short-range wireless connection is greater than or equal to the second threshold.

Clause 11: The method of any of Clauses 1-10, wherein the indication of the signal strength associated with the short-range wireless connection comprises a notification of a signal strength event.

Clause 12: The method of Clause 11, wherein the signal strength event occurs as a result of at least one of: a received signal strength indicator (RSSI) associated with the one or more information packets received from the analyte sensor system being below a signal strength threshold; a bit error rate (BER) associated with the one or more information packets received from the analyte sensor system being greater than or equal to a BER threshold; or a number of the one or more information packets being below a threshold number for a period of time.

Clause 13: The method of any of Clauses 11-12, wherein the notification includes a time period indicating when the signal strength event occurred.

Clause 14: The method of any of Clauses 11-13, wherein the notification includes location information of the display device indicating where the display device was located when the signal strength event occurred.

Clause 15: The method of any of Clauses 11-14, wherein the notification prompts the user to take corrective action to avoid or reduce occurrence of future signal strength events.

Clause 16: The method of any of Clauses 11-15, wherein: the notification includes an indication of a corrective action for the user to take; and the indication of the corrective action comprises at least one of: an indication for the user to reposition the display device; or an indication for the user to leave an area having an interference level, caused by transmissions associated with other devices, above a threshold.

Clause 17: The method of any of Clauses 11-16, wherein providing the indication of the signal strength associated with the short-range wireless connection comprises: determining that the signal strength associated with the short-range wireless connection is below a threshold signal strength; and providing the notification of the signal strength event to the user when the signal strength is below the threshold signal strength for a threshold amount of time.

Clause 18: The method of Clause 17, wherein the threshold amount of time is configurable by the user of the display device.

Clause 19: The method of any Clauses 11-18, wherein the notification of the signal strength event comprises a notification of a signal strength event predicted to occur in the future.

Clause 20: The method of Clause 19, further comprising determining the signal strength event predicted to occur in the future based on a plurality of signal strength events that have occurred in the past.

Clause 21: The method of Clause 20, wherein determining the signal strength event predicted to occur in the future based on the plurality of signal strength events that have occurred in the past comprises determining that the signal strength events in the plurality of signal strength events that have occurred in the past have occurred: at approximately a same time each day; at approximately regular intervals; or in a same approximate location.

Clause 22: The method of any of Clauses 1-21, wherein: the indication of the signal strength associated with the short-range wireless connection comprises an alert of a signal strength event; and the alert includes at least one of an auditory alert, a vibratory alert, or a light emitting diode (LED) alert.

Clause 23: The method of Clause 22, wherein providing the indication of the signal strength comprises providing the alert to the user when the signal strength reaches a certain signal strength threshold.

Clause 24: The method of Clause 23, wherein the certain signal strength threshold a minimum signal strength threshold in which communication between the analyte sensor system and the display device is still possible on the short-range wireless connection.

Clause 25: The method of Clause 24, wherein providing the alert to the user when the signal strength reaches a certain signal strength threshold provides the user with an amount of time to take corrective action before communication between the analyte sensor system and the display device is no longer possible on the short-range wireless connection.

Clause 26: The method of any of Clauses 23-25, further comprising transmitting control information to the analyte sensor system, instructing the analyte sensor system to provide the alert to the user.

Clause 27: The method of Clause 26, wherein: the control information instructing the analyte sensor system to provide the alert to the user prompts the user to look at a display of the display device; and the method further comprises providing, on the display of the display device, additional information associated with the signal strength event including at least one of: a time period associated with the signal strength event; location information indicating a location of the display device when the signal strength event occurred; or one or more corrective actions for the user to take to avoid future signal strength events or to avoid loss of the short-range wireless connection.

Clause 28: The method of any of Clauses 23-27, further comprising providing the user with an option to silence alarms of signal strength events for a period of time.

Clause 29: The method of any of Clauses 1-28, wherein providing the indication of the signal strength associated with the short-range wireless connection to the user comprises transmitting the indication of the signal strength to one or more second display devices associated with the user.

Clause 30: The method of Clause 29, wherein the one or more second display devices comprise at least one of a smart watch, smart glasses, or a virtual reality headset.

Clause 31: The method of any of Clauses 1-30, further comprising: determining one or more signal strength events associated with the short-range wireless connection based on the signal strength being less than or equal to a certain signal strength threshold; and in response to determining the one or more signal strength events, performing a transmission power negotiation procedure with the analyte sensor system.

Clause 32: The method of Clause 31, wherein performing the transmission power negotiation procedure comprises transmitting message to the analyte sensor system for increasing a transmission power for transmitting the one or more information packets.

Clause 33: The method of Clause 32, wherein the message indicates a specific value for the transmission power for the analyte sensor system to use or indicates the transmission power as an offset from a current transmission power of the one or more information packets.

Clause 34: The method of Clause 32, wherein: the message requests the analyte sensor system to transmit a power headroom report to the display device indicating an amount of transmission power that the analyte sensor system is capable of increasing to; and the method further comprises receiving the power headroom report from the analyte sensor system in response to the message.

Clause 35: The method of Clause 34, further comprising: determining the transmission power for the analyte sensor system to use for transmitting the one or more information packets based on the power headroom report; and transmitting, to the analyte sensor system, an indication of the determined transmission power for the analyte sensor system to use for transmitting the one or more information packets.

Clause 36: The method of Clause 35, further comprising receiving an acknowledgement message from the analyte sensor system acknowledging that the analyte sensor system will increase its transmission power based on the indication of the determined transmission power transmitted to the analyte sensor system.

Clause 37: The method of any of Clauses 31-36, further comprising predicting that the one or more signal strength events will occur at a particular time in the future based on one or more past signal strength events.

Clause 38: The method of Clause 37, wherein performing the transmission power negotiation procedure with the analyte sensor system comprises performing the transmission power negotiation procedure with the analyte sensor system prior to the signal strength event occurring at the particular time in the future.

Clause 39: The method of any of Clauses 37-38, wherein providing the indication of the signal strength associated with the short-range wireless connection comprises providing a notification to the user of the predicted one or more signal strength events.

Clause 40: The method of Clause 39, further comprising detecting that the predicted one or more signal strength events have occurred a threshold number of times after providing the notification to the user of the predicted one or more signal strength events, wherein performing the transmission power negotiation procedure with the analyte sensor system comprises performing the transmission power negotiation procedure with the analyte sensor system after detecting that the predicted one or more signal strength events have occurred the threshold number of times.

Clause 41: The method of any of Clauses 31-40, further comprising, after completion of the transmission power negotiation procedure, providing the user with an indication of at least one of that the transmission power negotiation procedure has been completed or that the transmission power for transmitting the one or more information packets has been increased.

Clause 42: The method of any of Clauses 1-41, wherein the one or more information packets includes analyte data indicating analyte levels of the user of the analyte sensor system.

Clause 43: The method of any of Clauses 1-42, wherein the short-range wireless connection comprises a Bluetooth connection or a Bluetooth Low Energy (BLE) connection.

Clause 44: An analyte monitoring system, comprising: an analyte sensor system and a display device configured to perform a method in accordance with and of Clauses 1-43.

Clause 45: A processing system of an apparatus, comprising: one or more processors individually or collectively configured to execute instructions stored on one or more memories and to cause the apparatus to perform a method in accordance with any one of Clauses 1-43.

Clause 46: An apparatus, comprising means for performing a method in accordance with any one of Clauses 1-43.

Clause 47: A non-transitory computer-readable medium comprising executable instructions that, when executed by one or more processors of an apparatus, cause the apparatus to perform a method in accordance with any one of Clauses 1-43.

Clause 48: A computer program product embodied on a computer-readable storage medium comprising code for performing a method in accordance with any one of Clauses 1-43.

### Additional Considerations

In this document, the terms “computer program medium” and “computer usable medium” and “computer readable medium”, as well as variations thereof, are used to generally refer to transitory or non-transitory media. These and other various forms of computer program media or computer usable/readable media may be involved in carrying one or more sequences of one or more instructions to a processing device for execution. Such instructions embodied on the medium, may generally be referred to as “computer program code” or a “computer program product” or “instructions” (which may be grouped in the form of computer programs or other groupings). When executed, such instructions may enable a computing module, such as the analyte sensor system 208, display device 210 display device 602, and/or communications device 1000, circuitry related thereto, and/or a processor thereof or connected thereto to perform features or functions of the present disclosure as discussed herein (for example, in connection with methods described above and/or in the claims), including for example when the same is/are incorporated into a system, apparatus, device and/or the like.

Various embodiments have been described with reference to specific example features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense. It will be appreciated that, for clarity purposes, the above description has described embodiments with reference to different functional units. However, it will be apparent that any suitable distribution of functionality between different functional units may be used without detracting from the invention. For example, functionality illustrated to be performed by separate computing devices may be performed by the same computing device. Likewise, functionality illustrated to be performed by a single computing device may be distributed amongst several computing devices. Hence, references to specific functional units are only to be seen as references to suitable means for providing the described functionality, rather than indicative of a strict logical or physical structure or organization.

Although described above in terms of various example embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the present application, whether or not such embodiments are described and whether or not such features are presented as being a part of a described embodiment. Thus, the breadth and scope of the present application should not be limited by any of the above-described example embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide illustrative instances of the item in discussion, not an exhaustive or limiting list thereof; the terms “a” or “an” should be read as meaning “at least one,” “one or more” or the like; the term “set” should be read to include one or more objects of the type included in the set; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Similarly, the plural may in some cases be recognized as applicable to the singular and vice versa. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic, circuitry, or other components, may be combined in a single package or separately maintained and may further be distributed in multiple groupings or packages or across multiple locations.

Additionally, the various embodiments set forth herein are described in terms of example block diagrams, flow charts, and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration. Moreover, the operations and sub-operations of various methods described herein are not necessarily limited to the order described or shown in the figures, and one of skill in the art will appreciate, upon studying the present disclosure, variations of the order of the operations described herein that are within the spirit and scope of the disclosure. It will be understood that each block of the flowchart illustrations, and combinations of blocks in the flowchart illustrations, can be implemented by execution of computer program instructions. These computer program instructions may be loaded onto a computer or other programmable data processing apparatus (such as a controller, microcontroller, microprocessor or the like) in a sensor electronics system to produce a machine, such that the instructions which execute on the computer or other programmable data processing apparatus create instructions for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks presented herein.

It should be appreciated that all methods and processes disclosed herein may be used in any glucose or other analyte monitoring system, continuous or intermittent. It should further be appreciated that the implementation and/or execution of all methods and processes may be performed by any suitable devices or systems, whether local or remote. Further, any combination of devices or systems may be used to implement the present methods and processes.

In addition, the operations and sub-operations of methods described herein may be carried out or implemented, in some cases, by one or more of the components, elements, devices, modules, circuitry, processors, etc. of systems, apparatuses, devices, environments, and/or computing modules described herein and referenced in various of figures of the present disclosure, as well as one or more sub-components, elements, devices, modules, processors, circuitry, and the like depicted therein and/or described with respect thereto. In such instances, the description of the methods or aspects thereof may refer to a corresponding component, element, etc., but regardless of whether an explicit reference is made, one of skill in the art will recognize upon studying the present disclosure when the corresponding component, element, etc. may be used. Further, it will be appreciated that such references do not necessarily limit the described methods to the particular component, element, etc. referred to. Thus, it will be appreciated by one of skill in the art that aspects and features described above in connection with (sub-) components, elements, devices, modules, and circuitry, etc., including variations thereof, may be applied to the various operations described in connection with methods described herein, and vice versa, without departing from the scope of the present disclosure.

1. A method for communication by a display device in an analyte monitoring system, comprising:
establishing a short-range wireless connection with an analyte sensor system;
receiving one or more information packets from the analyte sensor system;
determining a signal strength associated with the short-range wireless connection based on the one or more information packets received from the analyte sensor system; and
providing, to a user of the analyte sensor system, an indication of the signal strength associated with the short-range wireless connection.

2. The method of claim 1, wherein:
determining the signal strength associated with the short-range wireless connection comprises performing received signal strength indicator (RSSI) measurements on the one or more information packets received from the analyte sensor system; and
the indication of the signal strength comprises the RSSI measurements of the one or more information packets.

3. The method of claim 1, wherein:
determining the signal strength associated with the short-range wireless connection comprises determining a bit error rate (BER) associated with the one or more information packets received from the analyte sensor system;
the indication of the signal strength is based on the BER associated with the one or more information packets received from the analyte sensor system; and
the indication of the signal strength is different for different BERs associated with the one or more information packets received from the analyte sensor system.

4. (canceled)

5. The method of claim 1, wherein:
determining the signal strength associated with the short-range wireless connection comprises determining a number of the one or more information packets received from the analyte sensor system in a period of time;
the indication of the signal strength is based on the number of the one or more information packets received from the analyte sensor system in the period of time; and
the indication of the signal strength is different for different numbers of the one or more information packets received from the analyte sensor system in the period of time.

6. (canceled)

7. The method of claim 1, wherein:
providing the indication of the signal strength comprises displaying, on a display of the display device, a number of shapes that corresponds to the signal strength;
wherein the shapes comprise at least one of bars, a circles, of squares, or other shapes for representing the signal strength; and
different numbers of the shapes correspond to different signal strengths.

8-9. (canceled)

10. The method of claim 7, wherein:
the number of shapes displayed on the display comprises a first number of shapes when the signal strength associated with the short-range wireless connection is below a first threshold;
the number of shapes displayed on the display comprises a second number of shapes, greater than the first number of shapes, when the signal strength associated with the short-range wireless connection is greater than or equal to the first threshold and less than a second threshold; and
the number of shapes displayed on the display comprises a third number of shapes, greater than the second number of shapes, when the signal strength associated with the short-range wireless connection is greater than or equal to the second threshold.

11. The method of claim 1, wherein the indication of the signal strength associated with the short-range wireless connection comprises a notification of a signal strength event.

12. The method of claim 11, wherein the signal strength event occurs as a result of at least one of:
a received signal strength indicator (RSSI) associated with the one or more information packets received from the analyte sensor system being below a signal strength threshold;
a bit error rate (BER) associated with the one or more information packets received from the analyte sensor system being greater than or equal to a BER threshold; or
a number of the one or more information packets being below a threshold number for a period of time.

13. The method of claim 11, wherein the notification includes a time period indicating when the signal strength event occurred.

14. The method of claim 11, wherein the notification includes location information of the display device indicating where the display device was located when the signal strength event occurred.

15. The method of claim 11, wherein the notification prompts the user to take corrective action to avoid or reduce occurrence of future signal strength events.

16. The method of claim 11, wherein:
the notification includes an indication of a corrective action for the user to take; and
the indication of the corrective action comprises at least one of:
an indication for the user to reposition the display device; or
an indication for the user to leave an area having an interference level, caused by transmissions associated with other devices, above a threshold.

17. The method of claim 11, wherein providing the indication of the signal strength associated with the short-range wireless connection comprises:
determining that the signal strength associated with the short-range wireless connection is below a threshold signal strength; and
providing the notification of the signal strength event to the user when the signal strength is below the threshold signal strength for a threshold amount of time.

18. The method of claim 17, wherein the threshold amount of time is configurable by the user of the display device.

19. The method of claim 11, wherein:
the notification of the signal strength event comprises a notification of a signal strength event predicted to occur in the future;
the method further comprises determining the signal strength event predicted to occur in the future based on a plurality of signal strength events that have occurred in the past; and
determining the signal strength event predicted to occur in the future based on the plurality of signal strength events that have occurred in the past comprises determining that the signal strength events in the plurality of signal strength events that have occurred in the past have occurred:
at approximately a same time each day;
at approximately regular intervals; or
in a same approximate location.

20-30. (canceled)

31. The method of claim 1, further comprising:
determining one or more signal strength events associated with the short-range wireless connection based on the signal strength being less than or equal to a certain signal strength threshold; and
in response to determining the one or more signal strength events, performing a transmission power negotiation procedure with the analyte sensor system.

32. The method of claim 31, wherein:
performing the transmission power negotiation procedure comprises transmitting message to the analyte sensor system for increasing a transmission power for transmitting the one or more information packets; and
the message indicates a specific value for the transmission power for the analyte sensor system to use or indicates the transmission power as an offset from a current transmission power of the one or more information packets.

33-36. (canceled)

37. The method of claim 31, further comprising predicting that the one or more signal strength events will occur at a particular time in the future based on one or more past signal strength events, wherein performing the transmission power negotiation procedure with the analyte sensor system comprises performing the transmission power negotiation procedure with the analyte sensor system prior to the signal strength event occurring at the particular time in the future.

38-43. (canceled)

44. An analyte monitoring system, comprising:
an analyte sensor system; and
a display device, wherein:
the analyte sensor system is configured to:
measure analyte levels associated with a user of the analyte sensor system; and
transmit one or more information packets to the display device; and
the display device is configured to:
establish a short-range wireless connection with the analyte sensor system;
receive the one or more information packets from the analyte sensor system;
determine a signal strength associated with the short-range wireless connection based on the one or more information packets received from the analyte sensor system; and
provide, to a user of the analyte sensor system, an indication of the signal strength associated with the short-range wireless connection.

45-61. (canceled)

62. A processing system for communication by a display device in an analyte monitoring system, comprising:
one or more processors individually or collectively configured to execute instructions stored on one or more memories and to cause the display device to:
establish a short-range wireless connection with an analyte sensor system;
receive one or more information packets from the analyte sensor system;
determine a signal strength associated with the short-range wireless connection based on the one or more information packets received from the analyte sensor system; and
provide, to a user of the analyte sensor system, an indication of the signal strength associated with the short-range wireless connection.

63-104. (canceled)
